(19) United States
(12) Patent Application Publication
(10) Pub. No.: US 2025/0172562 A1
(43) Pub. Date: May 29, 2025

(54) CONTACT-TYPE PATCHES FOR STAINING

(71) Applicant: The General Hospital Corporation, Boston, MA (US)

(72) Inventors: Hyungsoon Im, Peabody, MA (US); Ralph Weissleder, Peabody, MA (US); Hakho Lee, Acton, MA (US); Cesar M. Castro, Cambridge, MA (US)

(21) Appl. No.: 18/839,262

(22) PCT Filed: Feb. 16, 2023
(86) PCT No.: PCT/US2023/062720

§ 371 Date: Aug. 16, 2024

Related U.S. Application Data
(60) Provisional application No. 63/310,702, filed on Feb. 16, 2022.

(51) Int. Cl. G01N 33/574; G01N 33/542; G01N 33/544

(52) U.S. Cl. CPC G01N 33/57492; G01N 33/542; G01N 33/544

(57) ABSTRACT

The present disclosure relates to the use of a contact-type patch in a staining process. An exemplary method for quenching a fluorophore present in a biological sample includes contacting a surface of a quenching patch including a polymer-containing substrate and a quenching agent disposed within the substrate, with the biological sample.

### CLAIM OF PRIORITY

This application claims priority to U.S. Patent Application Ser. No. 63/310,702, filed on Feb. 16, 2022, the entire contents of which are hereby incorporated by reference.

### TECHNICAL FIELD

Described herein are contact-type patches and methods of use thereof in a staining process.

### BACKGROUND

With advances in precision medicine, molecular analysis of tumors is increasingly adapted in daily practice for cancer diagnosis, subtype classification, prognosis, therapeutic decisions, and response monitoring. For example, in breast cancer, the classification of molecular subtypes has become a key component to providing information on prognosis and treatment decisions since targeted therapies became available for patients with positive expressions of estrogen receptors (ERs), progesterone receptors (PRs), and human epidermal growth factor receptor 2 (HER2) in tumor tissues. Thus, tools that provide the molecular characterization of tumor and tumor-associated immune cells can greatly improve patient outcomes by enabling earlier, more precise clinical decision points. Immunohistochemistry (IHC) staining is commonly used to detect the presence of specific protein markers that could assist with tumor diagnosis and classification. However, chromogenic IHC is limited in multiplexed analysis, and is not suitable to detect multiple markers co-localized within a cell.

Multicolor immunofluorescence (IF) imaging is commonly used for multiplexed analysis of cellular markers in cytology samples and tissue sections (frozen or paraffin-embedded followed by antigen retrieval). This information can be used for heterogeneity analysis, spatial profiling, or medical diagnostics. Immunostaining is typically done in a solution of well-suspended cells. However, staining of tissue sections can be more challenging and often requires extensive optimization, expertise, and instrumentation. Furthermore, traditional methods can require multiple washing and staining steps that are lengthy and often expensive due to large antibody consumption. Conventional methods are also often inefficient because of the low effective diffusion caused by the low ratio of contact surface area to volume between reagents and tissues; this is typically compensated for by long staining time, for example, from hours to overnight.

Accordingly, there is a need for improved staining methods.

### SUMMARY

Provided herein is a method of quenching a fluorophore present in a biological sample, the method including contacting a surface of a quenching patch with the sample. The quenching patch includes a substrate containing a polymer, and a quenching agent disposed within the substrate.

Also provided herein is a method of staining a biological sample. The method includes contacting a surface of a first staining patch with the sample, the first staining patch including a substrate containing a polymer, and a first fluorophore-conjugated probe disposed within the substrate. The method includes removing the first staining patch to provide a first stained sample, and measuring a fluorescence intensity of the first stained sample. The method includes contacting a surface of a quenching patch with the first stained sample, the quenching patch including a substrate containing a substrate containing a polymer, and a quenching agent disposed within the substrate. The method includes removing the quenching patch to provide a quenched sample. The method includes contacting a surface of a second staining patch with the quenched sample, the second staining patch including a substrate containing a polymer, and a second fluorophore-conjugated probe disposed within the substrate. The method includes removing the second staining patch to provide a second stained sample, and measuring a fluorescence intensity of the second stained sample.

Also provided herein is a kit containing a precursor patch including a substrate containing a polymer, a first aqueous solution containing a first fluorophore-conjugated probe, a second aqueous solution containing a second fluorophore-conjugated probe, and a third aqueous solution containing a quenching agent.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic of staining according to an embodiment of the present disclosure.

FIG. 2 is an image showing the water contact angle of an embodiment of a contact-type patch described herein.

FIG. 3 is an image of stained BT474 breast cancer cells prepared according to an embodiment of the present disclosure. Antibodies against estrogen receptor (ER), progesterone receptor (PR), and human epidermal growth factor receptor 2 (HER2) were labeled different colors of fluorophores. Scale bar: 20 μm.

FIG. 4 is an image showing an embodiment of a contact-type patch described herein embedded into a holder, and inset, an embodiment of a staining patch described herein.

FIG. 5 is a scanning electron micrograph (SEM) image of an embodiment of a contact-type patch described herein. Scale bar: 500 nm.

FIG. 6 is a set of images showing the water contact angle of certain embodiments of a contact-type patch described herein.

FIG. 7 is a set of images of BT474 breast cancer cells stained for HER2, prepared according to an embodiment of the present disclosure.

FIG. 8 is a graph showing fluorescence intensities of cells (left bar, in each set) and background (right bar, in each set) for stained samples prepared according to certain embodiments of the present disclosure.

FIG. 9 is a graph showing fluorescence intensities of cells (left bar, in each set) and background (right bar, in each set) for stained samples prepared according to certain embodiments of the present disclosure.

FIG. 10 is a graph comparing signal-to-background ratios of stained samples prepared according to certain embodiments of the present disclosure.

FIG. 11 is a set of cryo-SEM images, images showing the water contact angle, and images showing the dissipation depth of certain contact-type patches described herein. Scale bar in SEM images: 1 μm (first row) and 500 nm (second row).

FIG. 12 is a set of fluorescence images of BT474 breast cancer cells stained according to an embodiment of the present disclosure.

FIG. 13 is a graph showing measured fluorescence intensities of cells and background of samples stained according to an embodiment of the present disclosure, at different time points.

FIG. 14 is a graph comparing measured fluorescence of cells stained according to certain embodiments of the present disclosure.

FIG. 15 is a graph comparing fluorescence intensities of surface (HER2) and intracellular (PR) markers of BT474 breast cancer cells stained according to an embodiment of the present disclosure (left bar, in each set), and BT474 breast cancer cells stained using solution-based methods (center and right bars, in each set).

FIG. 16 is a graph comparing staining uniformity for cells stained according to an embodiment of the present disclosure (left set of bars) and for cells stained using solution-based methods (right set of bars).

FIG. 17 is a set of images showing the labelling uniformity of cells stained according to an embodiment of the present disclosure.

FIG. 18 is a schematic of immunocycling according to an embodiment of the present disclosure.

FIG. 19 is a set of fluorescence images and corresponding intensity profiles for cells stained according to an embodiment of the present disclosure, before and after quenching.

FIG. 20 is a set of fluorescence images of BT474 breast cancer cells stained for 9 different biomarkers in 3 cycles, according to an embodiment of the present disclosure. Scale bar: 50 μm.

FIG. 21 is a set of fluorescence images of MCF7 breast cancer cells stained for 9 different biomarkers in 3 cycles, according to an embodiment of the present disclosure. Scale bar: 50 μm.

FIG. 22 is a set of fluorescence images of HCC1954 breast cancer cells stained for 9 different biomarkers in 3 cycles, according to an embodiment of the present disclosure. Scale bar: 50 μm.

FIG. 23 is a set of fluorescence images of MDA-MB-231 breast cancer cells stained for 9 different biomarkers in 3 cycles, according to an embodiment of the present disclosure. Scale bar: 50 μm.

FIG. 24 is a set of graphs comparing cellular fluorescence intensities (mean+standard deviation) of BT474, MCF7, HCC1954, and MDA-MB-231 breast cancer cells stained for 9 different biomarkers in 3 cycles, according to an embodiment of the present disclosure.

FIG. 25 is a set of fluorescence images of tissue sections stained overnight using a solution-based method, and tissue sections stained according to certain embodiments of the present disclosure.

FIG. 26 is a set of graphs comparing mean fluorescence intensities of target biomarkers HER2, PR, ER, and Ki-67, of tissue samples stained according to certain embodiments of the present disclosure (for 15-120 min) and tissue sections stained overnight using a solution-based method.

FIG. 27 is a set of fluorescence images of breast cancer tissues stained against HER2, PR, and ER according to an embodiments of the present disclosure. Positivity of those markers in pathology reports are labeled on each image. Scale bar: 250 μm (whole tissue core) and 40 μm (zoomed-in images).

FIG. 28 is a set of graphs showing percentages of positive cells for HER2, PR, and ER in respective marker positive and negative tissue samples stained according to an embodiment of the present disclosure.

FIG. 29 is a heatmap showing percentages of HER2, PR, and ER positive cells in 12 tissue samples stained according to an embodiment of the present disclosure (p<0.01, unpaired t-test).

FIG. 30 is a heatmap showing the positivity of HER2, PR, and ER in pathology reports for 12 tissue samples stained according to an embodiment of the present disclosure.

FIG. 31 is a set of images showing tissue samples stained in cycle 1 for HER2, PR, and ER according to an embodiment of the present disclosure, (a) before and (b) after quenching, and (c)-(d) stained in cycle 2 for Ki-67. Numbers in (d) represent the Ki-67 positivity in pathology reports. Scale bar: 250 μm (whole tissue core) and 40 μm (zoomed-in images).

FIG. 32 is a set of images of breast cancer tissue samples with different antigen (HER2, PR, ER, and Ki-67) expression levels, profiled according to an embodiment of the present disclosure. Scale bar: 40 μm.

FIG. 33 is a graph comparing the effectiveness of certain quenching agents (quenched 30 min. in solution, 200 ms imaging).

FIG. 34 is a graph comparing the effectiveness of certain concentrations of a hydrogen peroxide quenching agent (quenched 30 min. in solution, 200 ms imaging).

FIG. 35 is a graph comparing the effectiveness of quenching according to certain embodiments of the present disclosure.

FIG. 36 is a set of fluorescence images of un-blocked cells, cells blocked using solution-based methods, and cells blocked using an embodiment of a contact-type patch described herein, after staining.

FIG. 37 is a graph comparing the normalized fluorescence intensity of un-blocked cells, cells blocked using solution-based methods, and cells blocked using an embodiment of a contact-type patch described herein, after staining.

FIG. 38 is a graph comparing the signal-to-background ratio of un-blocked cells, cells blocked using solution-based methods, and cells blocked using an embodiment of a contact-type patch described herein, after staining.

FIG. 39 is a set of fluorescence images of un-permeabilized cells, cells permeabilized using solution-based methods, and cells permeabilized using an embodiment of a contact-type patch described herein, after staining.

FIG. 40 is a graph comparing the signal-to-background ratio of un-permeabilized cells, cells permeabilized using solution-based methods, and cells permeabilized using an embodiment of a contact-type patch described herein, after staining.

### DETAILED DESCRIPTION

The present disclosure relates to the use of a contact-type patch in a staining process. In some embodiments, the staining is immunostaining. In some embodiments of the methods described herein, the contact-type patches can quickly and efficiently deliver a staining reagent to a biological sample. In some embodiments, the methods can be performed without the need for washing or other processing steps. In some embodiments, the methods can be performed using relatively little staining reagent. In some embodiments, the methods can include two or more cycles of fluorescence imaging.

### Contact-Type Patches

Provided herein are patches that include a polymer-containing substrate. In some embodiments, the polymer includes a chemically cross-linked polymer. In some embodiments, the polymer contains one or more monomer units selected from vinyl alcohol, ethylene glycol, ethylene oxide, 2-hydroxyethyl methacrylate, acrylic acid, and acrylamide monomer units.

In some embodiments, the polymer includes covalently cross-linked polyacrylamide, e.g., containing acrylamide and bis-acrylamide monomer units. For example, in some embodiments, the polymer is the product of polymerizing a mixture of acrylamide and bis-acrylamide monomer units, e.g., present in a molar ratio of about 20:1 to about 60:1, or about 30:1 to about 50:1.

In some embodiments, the polymer includes a physically cross-linked polymer. In some embodiments, the polymer includes one or more polymers selected from polysaccharides, polynucleotides, and polypeptides. In some embodiments, the polymer includes a polysaccharide such as cellulose, starch, agarose, chitin, chitosan, carrageenan, alginate, dextran, pullulan, or pectin. In some embodiments, the polymer includes physically cross-linked agarose.

In some embodiments, the patch contains about 4 wt % to about 12 wt % of the polymer. For example, in some embodiments, the patch contains about 4 wt % to about 10wt %, or about 4 wt % to about 9 wt %, or about 6 wt % to about 12 wt %, or about 6 wt % to about 10 wt %, or about 6 wt % to about 9 wt %, or about 7 wt % to about 12 wt %, or about 7 wt % to about 10 wt %, or about 7 wt % to about 9 wt % of the polymer. In some embodiments, the patch contains about 6 wt %, or about 7 wt %, or about 8 wt %, or about 9 wt %, or about 10 wt % of the polymer.

In some embodiments, the patch contains about 88 wt % to about 96 wt % of water. For example, in some embodiments, the patch contains about 88 wt % to about 94 wt %, or about 88 wt % to about 93 wt %, or about 90 wt % to about 96 wt %, or about 90 wt % to about 94 wt %, or about 90 wt % to about 93 wt %, or about 91 wt % to about 96 wt %, or about 91 to about 94 wt %, or about 91 wt % to about 93 wt % of water. In some embodiments, the patch contains about 90 wt %, or about 91 wt %, or about 92 wt %, or about 93 wt %, or about 94 wt % of water.

In some embodiments, the polymer makes up at least a portion of a polymer network containing a plurality of pores. As used herein, the term “polymer network” refers to a three-dimensional structure including chemically or physically cross-linked polymers. Such polymer networks can also be referred to as “hydrogels,” e.g., where the polymer network is hydrophilic and contains a relatively large amount of water.

In some embodiments, an average pore size of the plurality of pores is about 20 nm to about 75 nm. For example, in some embodiments, the average pore size of the plurality of pores is about 20 nm to about 65 nm, or about 20 nm to about 60 nm, or about 25 nm to about 75 nm, or about 25 nm to about 65 nm, or about 25 nm to about 60 nm, or about 30 nm to about 75 nm, or about 30 nm to about 65 nm, or about 30 nm to about 60 nm. In some embodiments, the average pore size of the plurality of pores is about 30 nm, or about 35 nm, or about 40 nm, or about 45 nm, or about 50 nm, or about 55 nm, or about 60 nm.

In some embodiments, the plurality of pores is present within a microstructure of the polymer network. For example, in some embodiments, the polymer network contains a honeycomb microstructure, e.g., having an average unit cell area of 0.03 μm² to about 1 μm², or about 0.1 μm² to about 0.25 μm², and, present within the honeycomb microstructure, the plurality of pores, e.g., having an average pore size of about 20 nm to about 75 nm, or about 30 nm to about 65 nm.

In some embodiments, the patch contains a surfactant disposed within the substrate. In some embodiments, the polymer makes up at least a portion of a polymer network containing a plurality of pores, and the surfactant is disposed within at least a portion of the plurality of pores. In some embodiments, the surfactant is a nonionic surfactant. In some embodiments, the surfactant is selected from a polysorbate, an octylphenol ethoxylate, a saponin, formaldehyde, methanol, and mixtures thereof. In some embodiments, the surfactant is selected from Tween 20, Triton X-100, and mixtures thereof.

In some embodiments, the patch includes about 0.01 wt % to about 2 wt % of the surfactant. For example, in some embodiments, the patch includes 0.01 wt % to about 1 wt %, or about 0.01 wt % to about 0.5 wt %, or about 0.01 wt % to about 0.25 wt %, or about 0.025 wt % to about 2 wt %, or about 0.025 wt % to about 1 wt %, or about 0.025 wt % to about 0.5 wt %, or about 0.025 wt % to about 0.25 wt %, or about 0.05 wt % to about 2 wt %, or about 0.05 wt % to about 1 wt %, or about 0.05 wt % to about 0.5 wt %, or about 0.05 wt % to about 0.25 wt % of the surfactant. In some embodiments, the patch includes about 0.05 wt %, or about 0.075 wt %, or about 0.1 wt %, or about 0.125 wt %, or about 0.15 wt %, or about 0.2 wt %, or about 0.25 wt % of the surfactant.

Such patches can be formed, by example, by gelling a precursor solution including water, one or more polymer network precursors, and the surfactant. For example, in some embodiments, the patch contains the product of polymerizing a mixture of acrylamide and bis-acrylamide present in an aqueous solution further including the surfactant. In certain such embodiments, the acrylamide and bis-acrylamide are present in a molar ratio of about 20:1 to about 60:1, or about 30:1 to about 50:1.

In some embodiments, the patch contains a blocking agent disposed within the substrate. In some embodiments, the polymer makes up at least a portion of a polymer network containing a plurality of pores, and the blocking agent is disposed within at least a portion of the plurality of pores. In some embodiments, the blocking agent is selected from a bovine serum albumin, fetal bovine serum, goat serum, steelhead salmon serum, non-fat milk, SuperBlock™ (Thermo Scientific), AdvanBlock™ (Advantsta Inc.) and mixtures thereof. In some embodiments, the blocking agent is bovine serum albumin.

In some embodiments, the patch includes about 0.1 wt % to about 5 wt % of the blocking agent. For example, in some embodiments, the patch includes about 0.1 wt % to about 3 wt %, or about 0.1 wt % to about 2 wt %, or about 0.1 wt % to about 1.5 wt %, or about 0.25 wt % to about 5 wt %, or about 0.25 wt % to about 3 wt %, or about 0.25 wt % to about 2 wt %, or about 0.5 wt % to about 1.5 wt % of the blocking agent. In some embodiments, the patch includes about 0.5 wt %, or about 0.75 wt %, or about 1 wt %, or about 1.25 wt %, or about 1.5 wt %, or about 1.75 wt %, or about 2 wt % of the blocking agent.

Such patches can be formed, by example, by gelling a precursor solution including water, one or more polymer network precursors, and the blocking agent. For example, in some embodiments, the patch contains the product of polymerizing a mixture of acrylamide and bis-acrylamide present in an aqueous solution further including the blocking agent. In certain such embodiments, the acrylamide and bis-acrylamide are present in a molar ratio of about 20:1 to about 60:1, or about 30:1 to about 50:1. In certain embodiments, the aqueous solution further includes a surfactant, e.g., as otherwise described herein.

In some embodiments, a water contact angle of a surface of the patch (e.g., a contact surface of the patch) is less than about 60°. For example, in some embodiments, a water contact angle of a surface of the patch is less than about 50°, or less than about 40°, or less than about 30°, or less than about 20°, or less than about 17.5°, or less than about 15°, or less than about 12.5°, or less than about 10°. In some embodiments, a water contact angle of a surface of the patch is about 5° to about 20°, or about 5° to about 17.5°, or about 5° to about 15°, or about 5° to about 12.5°, or about 5° to about 10°, or about 7.5° to about 15°, or about 7.5° to about 12.5°, or about 7.5° to about 10°. In some embodiments, a water contact angle of a surface of the patch is about 6°, or about 7°, or about 8°, or about 9°, or about 10°. In some embodiments, a water contact angle of the surface of the patch is less than a water contact angle of a surface of a biological sample contacted with the patch.

In some embodiments, the patches as otherwise described herein can be “precursor patches” substantially free from staining reagents. In other embodiments, the patches as otherwise described herein can be “quenching patches” containing an oxidizing agent, or “staining patches” containing a fluorophore-conjugated probe.

In some embodiments, the quenching patch includes a quenching agent disposed within the substrate. In some embodiments, the polymer makes up at least a portion of a polymer network containing a plurality of pores, and the quenching agent is disposed within at least a portion of the plurality of pores. In some embodiments, the quenching agent includes a compound capable of oxidizing a fluorophore. For example, in some embodiments, the quenching agent includes a peroxide group, a periodate group, or a mixture thereof. In some embodiments, the quenching agent includes hydrogen peroxide. In some embodiments, the oxidizing agent includes sodium periodate.

In some embodiments, the polymer, e.g., making up at least a portion of a polymer network, contains the quenching agent. For example, in some embodiments, the polymer contains a peroxide group or a periodate group. In some embodiments, the polymer is capable of generating hydrogen peroxide. For example, in some embodiments, the substrate includes a hydrogen peroxide-generating hydrogel.

In some embodiments, the quenching agent includes a compound capable of absorbing excitation energy from a fluorophore. In some embodiments, the quenching agent includes an iodide, an acrylamide, a black hole quencher, a QSY quencher, a DABCYL quencher, malachite green, gold nanoparticles, graphene, or any mixture thereof.

Quenching patches described herein can be formed, for example, by providing a precursor patch as otherwise described herein, and then applying an aqueous solution containing the quenching agent to a surface (e.g., a contact surface) of the precursor patch. Applying the aqueous solution can include, for example, dropping the solution onto the surface of the precursor patch, or dipping the surface of the precursor patch into a container of the solution.

In some embodiments, the aqueous solution contains about 0.5 wt % to about 10 wt %, or about 0.5 wt % to about 7.5 wt %, or about 0.5 wt % to about 5 wt %, or about 1 wt % to about 10 wt %, or about 1 wt % to about 7.5 wt %, or about 1 wt % to about 5 wt %, or about 2 wt % to about 10 wt %, or about 2 wt % to about 7.5 wt %, or about 2 wt % to about 5 wt % of the quenching agent. In some embodiments, the aqueous solution contains about 1 wt %, or about 2 wt %, or about 3 wt %, or about 4 wt %, or about 5 wt % of the quenching agent. In some embodiments, a pH of the aqueous solution is greater than about 7, or greater than about 8, or greater than about 9. In some embodiments, a pH of the aqueous solution is about 7 to about 11, or about 7 to about 10, or about 8 to about 11, or about 8 to about 10. In some embodiments, a pH of the aqueous solution is about 7.5, or about 8, or about 8.5, or about 9, or about 9.5, or about 10. In some embodiments, a pH of the aqueous solution is less than about 7, e.g., less than about 6, or less than about 5.

In some embodiments, about 1 μL to about 20 μL of the aqueous solution containing the quenching agent can be applied per square centimeter of the surface of the precursor patch, to form the quenching patch. For example, in some embodiments, the quenching patch can be formed by applying about 1 μL to about 15 μL, or about 1 μL to about 10 μL, or about 2 μL to about 20 μL, or about 2 μL to about 15 μL, or about 2 μL to about 10 μL of the aqueous solution containing the quenching agent per square centimeter of the surface of the precursor patch.

In some embodiments, the staining patch contains a fluorophore-conjugated probe disposed within the substrate. In some embodiments, the polymer makes up at least a portion of a polymer network containing a plurality of pores, and the fluorophore-conjugated probe is disposed within at least a portion of the plurality of pores. In some embodiments, the fluorophore includes a fluorescence-generating protein or a fluorescence-generating small molecule. For example, in some embodiments, the fluorophore includes a coumarin dye, a rhodamine dye, a cyanine dye, or a xanthene dye. In some embodiments, the fluorophore includes a cyanine dye, e.g., an Alexa Fluor 488, 555, or 647 dye (Invitrogen).

In some embodiments, the probe includes a nucleic acid capable of binding a biomarker, e.g., a nucleic acid marker. In some embodiments, the probe includes an antibody capable of binding a biomarker, e.g., a protein marker. The biomarker can be a cellular or molecular target relevant to, for example, cancers, neurodegenerative diseases, blood/vascular diseases, infection, inflammation, or wound healing. In some embodiments, the probe includes an antibody capable of binding a biomarker selected from cancer biomarkers. For example, in some embodiments, the probe includes an antibody capable of binding a biomarker selected from EpCAM, EGFR, MUC-1, HER2, PR, ER, Ki-67, CD45, and DAPI. In some embodiments, the staining patch contains two or more fluorophore-conjugated probes, e.g., a first, second, and third fluorophore-conjugated probe, each disposed within the substrate.

Staining patches described herein can be formed, for example, by providing a precursor patch as otherwise described herein, and then applying an aqueous solution containing the fluorophore-conjugated probe to a surface (e.g., a contact surface) of the precursor patch. Applying the aqueous solution can include, for example, dropping the solution onto the surface of the precursor patch, or dipping the surface of the precursor patch into a container of the solution.

In some embodiments, the aqueous solution contains about 10 ng/ml to 1 mg/mL, or about 10 ng/mL to about 500 μg/mL, or about 100 ng/mL to 1 mg/mL, or about 100 ng/mL to about 500 μg/mL, or about 1 μg/mL to 1 mg/mL, or about 1 μg/mL to about 500 μg/mL, or about 10 μg/mL to 1 mg/mL, or about 10 μg/mL to about 500 μg/mL of the fluorophore-conjugated probe. In some embodiments, a pH of the aqueous solution is about 7.

In some embodiments, about 1 μL to about 20 μL of the aqueous solution containing the fluorophore-conjugated probe can be applied per square centimeter of the surface of the precursor patch, to form the staining patch. For example, in some embodiments, the quenching patch can be formed by applying about 1 μL to about 15 μL, or about 1 μL to about 10 μL, or about 2 μL to about 20 μL, or about 2 μL to about 15 μL, or about 2 μL to about 10 μL of the aqueous solution containing the fluorophore-conjugated probe per square centimeter of the surface of the precursor patch.

### Quenching Methods

Also provided herein are methods for quenching a fluorophore present in a biological sample. For example, in some embodiments, the fluorophore is conjugated to a probe bound to a biomarker present in the sample, e.g., a stained sample. In some embodiments, quenching the fluorophore using a quenching patch as described herein can provide a quenched sample suitable for further staining without additional processing such as washing.

The method includes contacting a surface of a quenching patch including a polymer-containing substrate and a quenching agent disposed within the substrate, with the fluorophore-containing biological sample. The quenching patch can be any quenching patch described herein. In some embodiments, the quenching agent includes a compound capable of oxidizing a fluorophore. For example, in some embodiments, the quenching agent includes a peroxide group, a periodate group, or a mixture thereof. In some embodiments, the quenching agent includes hydrogen peroxide. In some embodiments, the oxidizing agent includes sodium periodate.

In some embodiments, the polymer, e.g., making up at least a portion of a polymer network, contains the quenching agent. For example, in some embodiments, the polymer contains a peroxide group or a periodate group. In some embodiments, the polymer is capable of generating hydrogen peroxide. For example, in some embodiments, the substrate contains a hydrogen peroxide-generating hydrogel.

In some embodiments, the quenching agent includes a compound capable of absorbing excitation energy from a fluorophore. In some embodiments, the quenching agent includes an iodide, an acrylamide, a black hole quencher, a QSY quencher, a DABCYL quencher, malachite green, gold nanoparticles, graphene, or any mixture thereof.

In some embodiments, contacting the surface of the quenching patch with the biological sample oxidizes at least 70%, or at least 75%, or at least 80%, or at least 85%, or at least 90%, or at least 95%, or at least 97.5%, or at least 98%, or at least 99% of the fluorophore present in the biological sample.

In some embodiments, the method includes removing the quenching patch to provide a quenched sample. In some embodiments, a fluorescence intensity of the quenched sample is less than 30%, or less than 25%, or less than 20%, or less than 15%, or less than 10%, or less than 5%, or less than 2.5%, or less than 2%, or less than 1% of the fluorescence intensity of the biological sample before contacting the quenching patch.

In some embodiments, a relatively large proportion of the quenching agent that does not quench the fluorophore present in the biological sample, e.g., by oxidizing the fluorophore, is retained in the quenching patch. In some embodiments, an amount of quenching agent present in the quenched sample is less than 1 wt %, or less than 0.5 wt %, or less than 0.25 wt %, or less than 0.1 wt % of the quenched sample. In some embodiments, the quenched sample is substantially free from quenching agent.

In some embodiments, the method includes providing a precursor patch including the polymer, and then applying an aqueous solution containing the quenching agent to a surface of the precursor patch to form the quenching patch. The precursor patch can be any precursor patch described herein.

In some embodiments, the method includes applying an aqueous solution containing about 0.5 wt % to about 10 wt %, or about 0.5 wt % to about 7.5 wt %, or about 0.5 wt % to about 5 wt %, or about 1 wt % to about 10 wt %, or about 1 wt % to about 7.5 wt %, or about 1 wt % to about 5 wt %, or about 2 wt % to about 10 wt %, or about 2 wt % to about 7.5 wt %, or about 2 wt % to about 5 wt % of the quenching agent to a surface of the precursor patch. In some embodiments, the aqueous solution contains about 1 wt %, or about 2 wt %, or about 3 wt %, or about 4 wt %, or about 5 wt % of the quenching agent. In some embodiments, a pH of the aqueous solution is greater than about 7, or greater than about 8, or greater than about 9. In some embodiments, a pH of the aqueous solution is about 7 to about 11, or about 7 to about 10, or about 8 to about 11, or about 8 to about 10. In some embodiments, a pH of the aqueous solution is about 7.5, or about 8, or about 8.5, or about 9, or about 9.5, or about 10. In some embodiments, a pH of the aqueous solution is less than about 7, e.g., less than about 6, or less than about 5.

In some embodiments, the method includes applying about 1 μL to about 20 μL of the aqueous solution containing the quenching agent per square centimeter of the surface of the precursor patch. For example, in some embodiments, the method includes applying about 1 μL to about 15 μL, or about 1 μL to about 10 μL, or about 2 μL to about 20 μL, or about 2 μL to about 15 μL, or about 2 μL to about 10 μL of the aqueous solution containing the quenching agent per square centimeter of the surface of the precursor patch.

In some embodiments, the fluorophore includes a fluorescence-generating protein or a fluorescence-generating small molecule. For example, in some embodiments, the fluorophore includes a coumarin dye, a rhodamine dye, a cyanine dye, or a xanthene dye. In some embodiments, the fluorophore includes a cyanine dye, e.g., an Alexa Fluor 488, 555, or 647 dye (Invitrogen). In some embodiments, the fluorophore is conjugated to a probe, e.g., including a nucleic acid or an antibody. In some embodiments, the probe includes an antibody capable of binding a biomarker selected from cancer biomarkers. In some embodiments, the probe includes an antibody capable of binding a biomarker selected from EpCAM, EGFR, MUC-1, HER2, PR, ER, Ki-67, CD45, and DAPI.

In some embodiments, the biological sample includes cells selected from tumor cells, non-tumor cells, immune cells, host cells, blood cells, and mixtures thereof. In some embodiments, the biological sample includes tumor cells. For example, in some embodiments, the biological sample includes tissue containing tumor cells. In some embodiments, the biological sample is fixed on a substrate, e.g., a glass slide.

### Staining Methods

Also provided herein are methods for staining a biological sample. The method includes contacting a surface of a first staining patch including a polymer-containing substrate and a first fluorophore-conjugated probe disposed within the substrate, with the biological sample. The method includes removing the first staining patch to provide a first stained sample, and measuring a fluorescence intensity of the first stained sample. The method includes contacting a surface of a quenching patch including a polymer-containing substrate and a quenching agent disposed within the substrate, with the first stained sample, and removing the quenching patch to provide a quenched sample. The method includes contacting a surface of a second staining patch including a polymer-containing substrate and a second fluorophore-conjugated probe disposed within the substrate, with the quenched sample. The method includes removing the second staining patch to provide a second stained sample, and measuring a fluorescence intensity of the second stained sample. In some embodiments, the process can be repeated, e.g., by contacting the second stained sample with a second quenching patch to provide a second quenched sample, and then contacting the second quenched sample with a third staining patch, two or more times, e.g., 6-10 times.

In some embodiments, the biological sample includes cells selected from tumor cells, non-tumor cells, immune cells, host cells, blood cells, and mixtures thereof. In some embodiments, the biological sample includes tumor cells. For example, in some embodiments, the biological sample includes tissue containing tumor cells. In some embodiments, the biological sample is fixed on a substrate, e.g., a glass slide.

In some embodiments, the method includes permeabilizing the biological sample, e.g., with a saponin or octylphenol ethoxylate, before contacting the first staining patch. In some embodiments, the method includes blocking the biological sample, e.g., with bovine serum albumin or goat serum, before contacting the first staining patch. In other embodiments, no permeabilization or blocking before contacting is necessary, e.g., where the first staining patch contains a surfactant and a blocking agent.

The first staining patch can be any staining patch described herein. In some embodiments, the first fluorophore-conjugated probe includes a fluorescence-generating protein or a fluorescence-generating small molecule. For example, in some embodiments, the first fluorophore-conjugated probe includes a coumarin dye, a rhodamine dye, a cyanine dye, or a xanthene dye. In some embodiments, the first fluorophore-conjugated probe includes a cyanine dye, e.g., an Alexa Fluor 488, 555, or 647 dye (Invitrogen). In some embodiments, the first fluorophore-conjugated probe includes a nucleic acid or an antibody. In some embodiments, the first fluorophore-conjugated probe includes an antibody capable of binding a biomarker selected from cancer biomarkers. In some embodiments, the first fluorophore-conjugated probe includes an antibody capable of binding a biomarker selected from EpCAM, EGFR, MUC-1, HER2, PR, ER, Ki-67, CD45, and DAPI.

In some embodiments, the method includes providing a precursor patch including the polymer, and then applying an aqueous solution including the first fluorophore-conjugated probe to a surface of the precursor patch to form the first staining patch. The precursor patch can be any precursor patch described herein.

In some embodiments, the aqueous solution contains about 10 ng/ml to 1 mg/mL, or about 10 ng/mL to about 500 μg/mL, or about 100 ng/ml to 1 mg/mL, or about 100 ng/ml to about 500 μg/mL, or about 1 μg/mL to 1 mg/mL, or about 1 μg/mL to about 500 μg/mL, or about 10 μg/mL to 1 mg/mL, or about 10 μg/mL to about 500 μg/mL of the fluorophore-conjugated probe. In some embodiments, a pH of the aqueous solution is about 7.

In some embodiments, the method includes applying about 1 μL to about 20 μL of the aqueous solution containing the first fluorophore-conjugated probe per square centimeter of the surface of the precursor patch. For example, in some embodiments, the method includes applying about 1 μL to about 15 μL, or about 1 μL to about 10 μL, or about 2 μL to about 20 μL, or about 2 μL to about 15 μL, or about 2 μL to about 10 μL of the aqueous solution containing the first fluorophore-conjugated probe per square centimeter of the surface of the precursor patch.

In some embodiments, the method includes contacting the biological sample with the first staining patch for about 30 sec. to about 60 min. For example, in some embodiments, the method includes contacting the biological sample with the first staining patch for about 30 sec. to about 30 min., or about 30 sec. to about 15 min., or about 1 min. to about 60 min., or about 1 min. to about 30 min., or about 1 min. to about 15 min., or about 2 min. to about 60 min., or about 2 min. to about 30 min., or about 2 min. to about 15 min. In some embodiments, the method includes contacting the biological sample with the first staining patch for about 1 min., or about 2 min., or about 3 min., or about 4 min., or about 5 min., or about 6 min., or about 7 min., or about 8 min., or about 9 min., or about 10 min.

In some embodiments, a relatively large proportion of the first fluorophore-conjugated probe that does not bind to an intended biomarker present in the biological sample (e.g., unbound, or non-specifically bound) is retained in the first staining patch. Accordingly, in some embodiments, upon removing the patch, relatively little of the first fluorophore-conjugated probe present in the first stained sample is not bound to the intended biomarker, and the first stained sample is suitable for fluorescence imaging without further processing (e.g., washing).

In some embodiments, at least 70 wt % of an amount of the first fluorophore-conjugated probe present in the first stained sample is bound to a first biomarker present in the biological sample. For example, in some embodiments, at least 75 wt %, or at least 80 wt %, or at least 85 wt %, or at least 90 wt %, or at least 95 wt % of the first fluorophore-conjugated probe present in the first stained sample is bound to a first biomarker present in the biological sample. In some embodiments, the first stained sample is substantially free from first fluorophore-conjugated probes that are not bound to the first biomarker.

In some embodiments, measuring a fluorescence intensity of the first stained sample includes fluorescence imaging the first stained sample, e.g., with an epifluorescence microscope.

The quenching patch can be any quenching patch described herein. In some embodiments, the method includes providing a precursor patch including the polymer, and then applying an aqueous solution containing the quenching agent to a surface of the precursor patch to form the quenching patch.

The precursor patch can be any precursor patch described herein. In some embodiments, the quenching agent includes a compound capable of oxidizing a fluorophore. For example, in some embodiments, the quenching agent includes a peroxide group, a periodate group, or a mixture thereof. In some embodiments, the quenching agent includes hydrogen peroxide. In some embodiments, the oxidizing agent includes sodium periodate.

In some embodiments, the polymer, e.g., making up at least a portion of a polymer network, contains the quenching agent. For example, in some embodiments, the polymer contains a peroxide group or a periodate group. In some embodiments, the polymer is capable of generating hydrogen peroxide. For example, in some embodiments, the substrate includes a hydrogen peroxide-generating hydrogel.

In some embodiments, the quenching agent includes a compound capable of absorbing excitation energy from a fluorophore. In some embodiments, the quenching agent includes an iodide, an acrylamide, a black hole quencher, a QSY quencher, a DABCYL quencher, malachite green, gold nanoparticles, graphene, or any mixture thereof.

In some embodiments, the method includes applying an aqueous solution containing about 0.5 wt % to about 10 wt %, or about 0.5 wt % to about 7.5 wt %, or about 0.5 wt % to about 5 wt %, or about 1 wt % to about 10 wt %, or about 1 wt % to about 7.5 wt %, or about 1 wt % to about 5 wt %, or about 2 wt % to about 10 wt %, or about 2 wt % to about 7.5 wt %, or about 2 wt % to about 5 wt % of the quenching agent to a surface of the precursor patch. In some embodiments, the aqueous solution contains about 1 wt %, or about 2 wt %, or about 3 wt %, or about 4 wt %, or about 5 wt % of the quenching agent. In some embodiments, a pH of the aqueous solution is greater than about 7, or greater than about 8, or greater than about 9. In some embodiments, a pH of the aqueous solution is about 7 to about 11, or about 7 to about 10, or about 8 to about 11, or about 8 to about 10. In some embodiments, a pH of the aqueous solution is about 7.5, or about 8, or about 8.5, or about 9, or about 9.5, or about 10. In some embodiments, a pH of the aqueous solution is less than about 7, e.g., less than about 6, or less than about 5.

In some embodiments, the method includes applying about 1 μL to about 20 μL of the aqueous solution containing the quenching agent per square centimeter of the surface of the precursor patch. For example, in some embodiments, the method includes applying about 1 μL to about 15 μL, or about 1 μL to about 10 μL, or about 2 μL to about 20 μL, or about 2 μL to about 15 μL, or about 2 μL to about 10 μL of the aqueous solution containing the quenching agent per square centimeter of the surface of the precursor patch.

In some embodiments, the method includes contacting the first stained sample with the quenching patch for about 1 min. to about 2 hr. For example, in some embodiments, the method includes contacting the first stained sample with the quenching patch for about 1 min. to about 1 hr., or about 1 min. to about 30 min., or about 2.5 min. to about 2 hr., or about 2.5 min. to about 1 hr., or about 2.5 min. to about 30 min., or about 5 min. to about 2 hr., or about 5 min. to about 1 hr., or about 5 min. to about 30 min. In some embodiments, the method includes contacting the first stained sample with the quenching patch for about 2.5 min., or about 5 min., or about 10 min., or about 15 min., or about 20 min.

In some embodiments, contacting the surface of the quenching patch with the first stained sample oxidizes at least 70%, or at least 75%, or at least 80%, or at least 85%, or at least 90%, or at least 95%, or at least 97.5%, or at least 98%, or at least 99% of the first fluorophore-conjugated probe present in the biological sample.

In some embodiments, a fluorescence intensity of the quenched sample is less than 30%, or less than 25%, or less than 20%, or less than 15%, or less than 10%, or less than 5%, or less than 2.5%, or less than 2%, or less than 1% of the fluorescence intensity of the first stained sample.

In some embodiments, a relatively large proportion of the oxidizing agent that does not quench the first fluorophore-conjugated probe, e.g., by oxidizing the first fluorophore-conjugated probe, is retained in the quenching patch. In some embodiments, an amount of quenching agent present in the quenched sample is less than 1 wt %, or less than 0.5 wt %, or less than 0.25 wt %, or less than 0.1 wt % of the quenched sample. In some embodiments, the quenched sample is substantially free from quenching agent.

The second staining patch can be any staining patch described herein. In some embodiments, the second fluorophore-conjugated probe includes a fluorescence-generating protein or a fluorescence-generating small molecule. For example, in some embodiments, the second fluorophore-conjugated probe includes a coumarin dye, a rhodamine dye, a cyanine dye, or a xanthene dye. In some embodiments, the second fluorophore-conjugated probe includes a cyanine dye, e.g., an Alexa Fluor 488, 555, or 647 dye (Invitrogen). In some embodiments, the second fluorophore-conjugated probe includes a nucleic acid or an antibody. In some embodiments, the second fluorophore-conjugated probe includes an antibody capable of binding a biomarker selected from cancer biomarkers. In some embodiments, the second fluorophore-conjugated probe includes an antibody capable of binding a biomarker selected from EpCAM, EGFR, MUC-1, HER2, PR, ER, Ki-67, CD45, and DAPI.

In some embodiments, an absorbance spectrum of the first fluorophore- conjugated probe and an absorbance spectrum of the second fluorophore-conjugated probe overlap. For example, in some embodiments, the first fluorophore-conjugated probe and the second fluorophore-conjugated probe each include the same dye, e.g., a cyanine dye, conjugated to antibodies capable of binding different biomarkers.

In some embodiments, the method includes providing a precursor patch including the polymer, and then applying an aqueous solution containing the second fluorophore-conjugated probe to a surface of the precursor patch to form the second staining patch. The precursor patch can be any precursor patch described herein.

In some embodiments, the aqueous solution contains about 10 ng/ml to 1 mg/mL, or about 10 ng/ml to about 500 μg/mL, or about 100 ng/ml to 1 mg/mL, or about 100 ng/mL to about 500 μg/mL, or about 1 μg/mL to 1 mg/mL, or about 1 μg/mL to about 500 μg/mL, or about 10 μg/mL to 1 mg/mL, or about 10 μg/mL to about 500 μg/mL of the fluorophore-conjugated probe. In some embodiments, a pH of the aqueous solution is about 7.

In some embodiments, the method includes applying about 1 μL to about 20 μL of the aqueous solution containing the second fluorophore-conjugated probe per square centimeter of the surface of the precursor patch. For example, in some embodiments, the method includes applying about 1 μL to about 15 μL, or about 1 μL to about 10 μL, or about 2 μL to about 20 μL, or about 2 μL to about 15 μL, or about 2 μL to about 10 μL of the aqueous solution containing the second fluorophore-conjugated probe per square centimeter of the surface of the precursor patch.

In some embodiments, the method includes contacting the quenched sample with the second staining patch for about 30 sec. to about 60 min. For example, in some embodiments, the method includes contacting the quenched sample with the second staining patch for about 30 sec. to about 30 min., or about 30 sec. to about 15 min., or about 1 min. to about 60 min., or about 1 min. to about 30 min., or about 1 min. to about 15 min., or about 2 min. to about 60 min., or about 2 min. to about 30 min., or about 2 min. to about 15 min. In some embodiments, the method includes contacting the quenched sample with the second staining patch for about 1 min., or about 2 min., or about 3 min., or about 4 min., or about 5 min., or about 6 min., or about 7 min., or about 8 min., or about 9 min., or about 10 min.

In some embodiments, a relatively large proportion of the second fluorophore-conjugated probe that does not bind to an intended biomarker present in the quenched sample (e.g., unbound, or non-specifically bound) is retained in the second staining patch. Accordingly, in some embodiments, upon removing the patch, relatively little of the second fluorophore-conjugated probe present in the second stained sample is not bound to the intended biomarker, and the second stained sample is suitable for fluorescence imaging without further processing (e.g., washing).

In some embodiments, at least 70 wt % of an amount of the second fluorophore-conjugated probe present in the first stained sample is bound to a second biomarker present in the biological sample. For example, in some embodiments, at least 75 wt %, or at least 80 wt %, or at least 85 wt %, or at least 90 wt %, or at least 95 wt % of the second fluorophore-conjugated probe present in the second stained sample is bound to a second biomarker present in the biological sample. In some embodiments, the second stained sample is substantially free from second fluorophore-conjugated probes that are not bound to the second biomarker.

In some embodiments, a contribution of the first fluorophore-conjugated probe to the fluorescence intensity of the second stained sample is no more than 20%. For example, in some embodiments, a contribution of the first fluorophore-conjugated probe to the fluorescence intensity of the second stained sample is no more than 15%, or no more than 10%, or no more than 5%, or no more than 2.5%, or no more than 2%, or no more than 1%.

In some embodiments, measuring a fluorescence intensity of the second stained sample includes fluorescence imaging the second stained sample, e.g., with an epifluorescence microscope.

### Staining Kits

Also provided herein are kits, e.g., for performing a staining method described herein. The kit includes a precursor patch including a polymer-containing substrate, a first aqueous solution containing a first fluorophore-conjugated probe, a second aqueous solution containing a second fluorophore-conjugated probe, and a third aqueous solution containing a quenching agent.

The precursor patch can be any precursor patch described herein. In some embodiments, the kit includes two or more precursor patches, e.g., three precursor patches.

The first fluorophore-conjugated probe can be any fluorophore-conjugated probe described herein. In some embodiments, the first aqueous solution contains about 10 ng/ml to 1 mg/mL, or about 10 ng/mL to about 500 μg/mL, or about 100 ng/ml to 1 mg/mL, or about 100 ng/ml to about 500 μg/mL, or about 1 μg/mL to 1 mg/mL, or about 1 μg/mL to about 500 μg/mL, or about 10 μg/mL to 1 mg/mL, or about 10 μg/mL to about 500 μg/mL of the first fluorophore-conjugated probe. In some embodiments, a pH of the first aqueous solution is about 7.

The second fluorophore-conjugated probe can be any fluorophore-conjugated probe described herein. In some embodiments, the second aqueous solution contains about 10 ng/ml to 1 mg/mL, or about 10 ng/ml to about 500 μg/mL, or about 100 ng/ml to 1 mg/mL, or about 100 ng/ml to about 500 μg/mL, or about 1 μg/mL to 1 mg/mL, or about 1 μg/mL to about 500 μg/mL, or about 10 μg/mL to 1 mg/mL, or about 10 ug/mL to about 500 ug/mL of the second fluorophore-conjugated probe. In some embodiments, a pH of the second aqueous solution is about 7.

The quenching agent can be any quenching agent described herein. In some embodiments, the third aqueous solution contains about 0.5 wt % to about 10 wt %, or about 0.5 wt % to about 7.5 wt %, or about 0.5 wt % to about 5 wt %, or about 1 wt % to about 10 wt %, or about 1 wt % to about 7.5 wt %, or about 1 wt % to about 5 wt %, or about 2 wt % to about 10 wt %, or about 2 wt % to about 7.5 wt %, or about 2 wt % to about 5 wt % of the quenching agent. In some embodiments, the third aqueous solution contains about 1 wt %, or about 2 wt %, or about 3 wt %, or about 4 wt %, or about 5 wt % of the quenching agent. In some embodiments, a pH of the third aqueous solution is greater than about 7, or greater than about 8, or greater than about 9. In some embodiments, a pH of the third aqueous solution is about 7 to about 11, or about 7 to about 10, or about 8 to about 11, or about 8 to about 10. In some embodiments, a pH of the third aqueous solution is about 7.5, or about 8, or about 8.5, or about 9, or about 9.5, or about 10. In some embodiments, a pH of the third aqueous solution is less than about 7, e.g., less than about 6, or less than about 5.

### Definitions

In this disclosure, the terms “a,” “an,” and “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed in this disclosure, and not otherwise defined, is for the purpose of description only and not of limitation. Any use of section headings is intended to aid reading of the document and is not to be interpreted as limiting; information that is relevant to a section heading may occur within or outside of that particular section.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (for example, 1%, 2%, 3%, and 4%) and the sub-ranges (for example, 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

The term “about” as used in the present disclosure can allow for a degree of variability in a value or range, for example, within 10%, within 5%, or within 1% of a stated value or of a stated limit of a range.

In the methods described in the present disclosure, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

As used herein, the term “quench,” used in reference to a fluorophore, refers to partial or even complete reduction of the ability of a fluorophore to emit light upon photoexcitation. For example, “quenching” can include inactivating” a fluorophore, e.g., by oxidizing the fluorophore. In another example, “quenching” can include coupling, e.g. by contacting, a fluorophore with a compound capable of absorbing excitation energy from the fluorophore.

As used herein, the term “monomer unit,” used in reference to a polymer, refers to a monomer, or residue of a monomer, that has been incorporated into at least a portion of the polymer.

### EXAMPLES

The invention is further described in the following examples, which do not limit the scope of the invention described in the claims.

### General Experimental Details

Hydrogel Fabrication: 8% acrylamide mixture solution (100 mL) was made in the following order. First, Milli-Q water (51.4 ml), 10x PBS (pH 7.4), 10% BSA (VWR, 10 ml), 10% TritonX-100 (Sigma-Aldrich, 1 ml, 0.1% in total) and 30% acrylamide mix (Bio-Rad, 37.5:1 ratio, 26.6 ml, 8% in total) were mixed in order. Second, 10% ammonium persulfate (Samchun Chemicals, 1 ml) and tetramethylethylenediamine (Sigma-Aldrich, 0.1 ml) were added to the mixture. The mixed solution was gently stirred for 3 minutes. The final 8% acrylamide mixture solution was pipetted into the mold and allowed to solidify. The incubated hydrogel patches were taken out with plastic frames together from the molds. Patches were vacuum sealed by aluminum foil until used.

Cell lines: 4 different breast cancer cell lines from the American Type Culture Collection (ATCC) were used for assay validation: MDA-MB-231, BT474, MCF7, and HCC1954. Culture media DMEM was used for MCF7 and MCA-MB-231, while RMPI-1640 for BT474 and HCC1954. In addition, the culture media were supplemented with 10% fetal bovine serum and penicillin-streptomycin (Cellgro). All cell lines were tested for mycoplasma contamination.

Human breast cancer tissue samples: Breast carcinoma (CSB0825P), PRA IHC control slides (CS-PRA/15) from StatLab, and breast cancer tissue with HER2 IHC Control slides (BR082e) from Biomax were used for characterization and optimization. Human breast cancer and normal tissue microarray slides (BC081120e & BC08116d) from US Biomax were used for breast cancer subtyping.

Antibodies: The following antibodies were used in the immunolabeling experiments: epithelial cell adhesion molecule (EpCAM) AF488 conjugate (324210, BioLegend), isotype mouse IgG AF488 conjugate (400329, BioLegend); epidermal growth factor receptor (EGFR) AF555 conjugate (5108s, Cell Signaling Technology), isotype rabbit IgG AF555 conjugate (3969s, Cell Signaling Technology); mucin-1 APC conjugate (355607, BioLegend), isotype mouse IgG APC conjugate (400120, BioLegend); human epidermal growth factor receptor 2 (HER2) AF488 conjugate (324410, BioLegend), isotype mouse IgG AF488 conjugate (400132, BioLegend); progesterone receptor PR AF555 conjugate (67242BC, Cell Signaling Technology); estrogen receptor ER AF647 conjugate (57761s, Cell Signaling Technology), isotype rabbit IgG AF647 conjugate (3452s, Cell Signaling Technology); CD45 AF488 conjugate (368535, BioLegend); Ki-67 AF647 conjugate (12075s, Cell Signaling Technology). For tissue staining, HER2 AF488 conjugate (45332BC, Cell Signaling Technology), Ki-67 AF647 conjugate (12075S, Cell Signaling Technology), PR AF555 conjugate (83787BC, Cell Signaling Technology) and ER AF647 conjugate (ab267512, Abcam) were used. Isotype control antibodies, AF488 conjugate, AF555 conjugate and AF647 conjugate (4340S, 3969S and 3452S respectively, Cell Signaling Technology) were used as negative control.

Cryo-focused ion beam/scanning electron microscope (FIB/SEM) imaging: The cryo-FIB/SEM imaging and analysis were performed by using Quanta 3D FEG (FEI, Netherland) with an Alto 2500 cryo-transfer system (Gatan, UK). The procedure of cryo sampling is as follows: (1) Sample mounting: the hydrogel substrates were mounted to a stub. (2) Slush freezing: the substrates were rapidly submerged into liquid nitrogen slush within 0.5 sec. Rapidly, the freezing chamber was evacuated. Then, the samples were transferred into a preparation chamber, which temperature was set at −210° C. (3) Vacuum transfer: The samples were transferred to the cold stage of ALTO 2500. (4) Etching: Ice formed during the rapid freezing was sublimated at −95° C. for 3 min. (5) Au/Pd coating: Au/Pd coating was done to prevent the charging effect. (6) Imaging and analysis: the Au/Pd coated samples were transferred into a FIB chamber, pre-evacuated, and precooled to a temperature of −190° C. The cryo-SEM image was acquired with a 5-keV electron beam of energy and an electron current of 47 pA. All images were taken at the same scale of 50,000× and 100,000×.

Contact angle measurement: Static contact angles of the hydrogels were measured by contact angle meter (ST-GTD-1016) from SurfaceTech Co., Ltd. Deionized water (5 μL) was loaded on the surface of hydrogel samples.

Immunocycling assay: Cells were first permeabilized using BD Perm/Wash buffer (554723, BD Biosciences) for 10 min. and then blocked using 2.5% BSA (37525, ThermoFisher Scientific) and 2.5% goat serum (5425S, Cell Signaling Technology) in BD Perm/Wash buffer for another 10 min. Cells were then centrifuged using Cytospin 4 (ThermoFisher Scientific) at 800 RPC for 7 min. onto a polylysine slide (10144-822 ,VWR International). For immunostaining, 3 μL of mixed antibody reagents were dispersed onto the hydrogel. The hydrogel was then applied onto the cells for 5 min. Afterward, the labeled cells were imaged. Gold antifade mountant with DAPI (P36935, ThermoFisher Scientific) was used to stain the nuclei. For fluorescence quenching, 200 μL of 5% hydrogen peroxide (H1009, Sigma-Aldrich) and 7.5% sodium bicarbonate (25-035-CI, Corning) in deionized water was dropped onto the cells. The quenching process was catalyzed by using an incandescent light (2,300 lumens, Philips). The immunostaining and quenching steps were repeated for subsequent cycles.

Tissue staining: The paraffin-embedded sections were deparaffinized and rehydrated through 2 changes of xylene, 2 changes of 100% ethyl alcohol, 1 change of 95%, 70%, 50% ethyl alcohol, for 5 min. each. After rising the slides with distilled water, heat-induced antigen retrieval was performed using citrate unmasking solution (14746S, Cell Signaling Technology, Danvers, MA), according to manufacturer's instruction. The sections were permeabilized with 0.3% TritonX-100 (X-100, Sigma-Aldrich) in PBS for 10 min. at room temperature. After being washed in PBS three times for 5 min. each, the sections were blocked with 4% normal goat serum (S-1000, Vector Laboratories, Inc.) in PBS for 1 hr. at room temperature. For characterization and optimization, HER2, PR, ER, and ki67 antibodies were applied individually using hydrogels (20 μl) and incubated for 15, 30, 45, 60 and 120 min. at room temperature or without hydrogels (200 μl) overnight at 4° C. Isotype control antibodies were incubated overnight at 4° C. as negative controls. After that, nuclei were counterstained with DAPI (D21490, ThermoFisher Scientific) for 5 min. at room temperature. After rinsing with PBS, the slides were cover-slipped using VECTASHIELD mounting medium (H-1000, Vector Laboratories, Inc.), and all the slides were analyzed by using an automated fluorescence microscope BX-63 (Olympus). The process was repeated with the optimized staining time of 30 min. for the human breast cancer and normal tissue microarray slides. In cycle 1, HER2, PR, and ER antibodies were mixed (20 μl) and stained using a single hydrogel. After imaging, the coverslips were removed in PBS, and fluorophores were inactivated in 4.5% H₂O₂ (H1009, Sigma-Aldrich)/24 mM NaOH (AC424330025, Fisher Scientific Acros) in PBS under white light for 1 hr. at room temperature. The slides were washed with PBS four times for 5 min. each and cover-slipped for image acquisition to quantify the fluorescence quenching results. Subsequently, the coverslips were removed and the slides were rinsed with PBS. In cycle 2, the sections were incubated with ki67 antibody using hydrogel (20 μl) for 30 min. at room temperature or without hydrogels overnight at 4° C. After rinsing with PBS three times for 5 min. each, the slides were cover-slipped and the sections were analyzed using BX-63.

Fluorescence imaging: Fluorescence images of cell samples were acquired using Andor Zyla 5.5 SCMOS camera on a Nikon eclipse Ti inverted automated epifluorescence microscope. Fluorescence images of tissue samples were acquired using Olympus BX-63 upright epifluorescence microscope.

Image analysis: Fluorescence images were analyzed by ImageJ v2.0.0. Statistical data was analyzed and plotted using GraphPad Prism 8.

Flow cytometry: BD LSRII flow cytometer (BD Biosciences) was used to measured fluorescence signals in cell samples for comparison and correlation. Data was analyzed using FlowJo v10.6.0 (Tree Star, Inc.).

### Example 1. Hydrogel Immunostaining

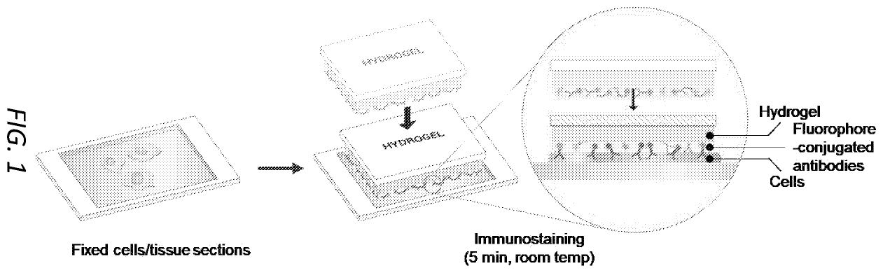

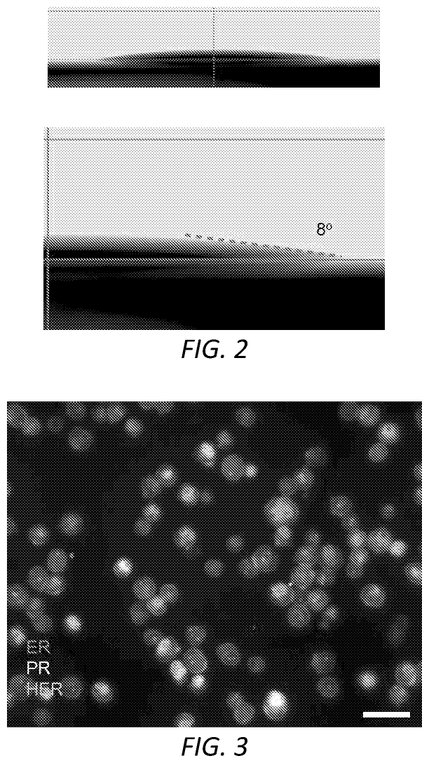

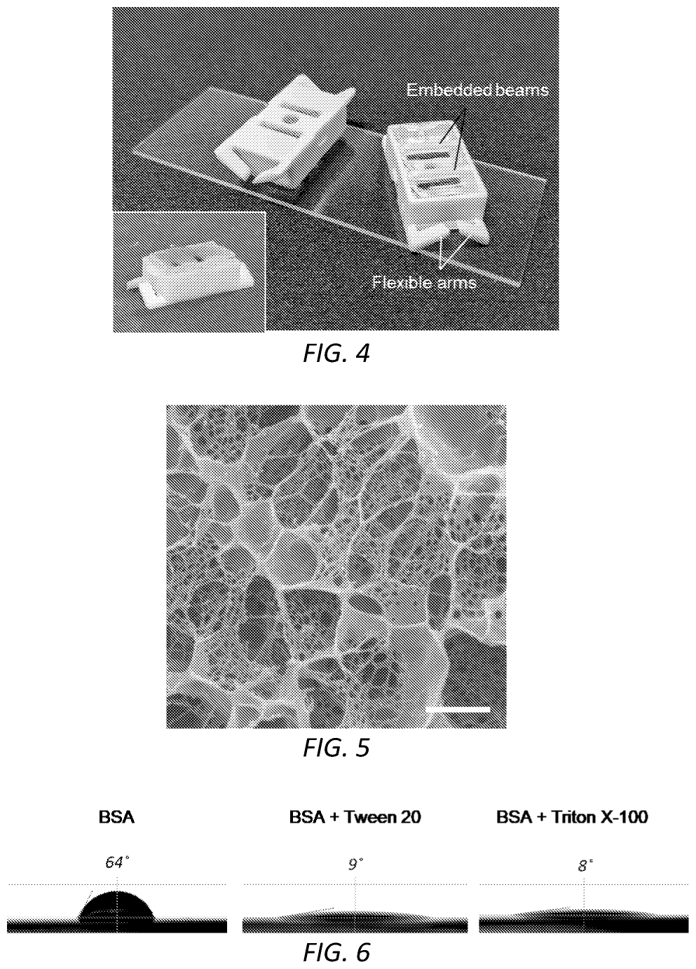

FIG. 1 shows a workflow of the hydrogel-based immunostaining for cells and tissue section samples. Once cells are placed on a glass slide, a hydrogel stamp wetted with a 3 μL antibody solution is placed on top of the fixed cells. When the hydrogel is placed on the cells, the increased surface area to volume ratio between the antibody solution and cells facilitates the staining process (5 min. for cells and 30 min. for tissue section, room temperature). Acrylamide was selected as a hydrogel material because of its hydrophilic property (FIG. 2). Because of the hydrophilicity of the hydrogel, the remaining antibody solution could be effectively removed when the hydrogel stamp was detached from the surface. In such a case, washing is not required after antibody incubation. The method is applicable for both surface and intracellular antigens. FIG. 3 shows a representative image of triple-positive (HER2+/ER+/PR+) breast cancer cells. It shows that HER2, PR, and ER were successfully immunolabeled in 5 min. and without any washing step. FIG. 4 shows fabricated hydrogels housed in a place holder and placed on top of cells on a glass slide. The hydrogel is precast in the holder with embedded beam structures to firmly hold the gel and flexible arms for easy handling. The hydrogel covers about a 2 cm² area (11 mm×18 mm). Larger sizes of hydrogel stamps can be fabricated easily for a larger staining area such as tissue samples. Without intending to be bound by any particular theory, because of the hydrophilicity of the gel, a small volume of antibody solution may easily be spread on the surface (Inset of FIG. 4). Scanning electron micrograph (SEM, FIG. 5) shows nanopores inside honeycomb-like hydrogel structures with an averaged pore size of 45±17 nm.

### Characterization of Hydrogel Concentrations and Surfactant Additives

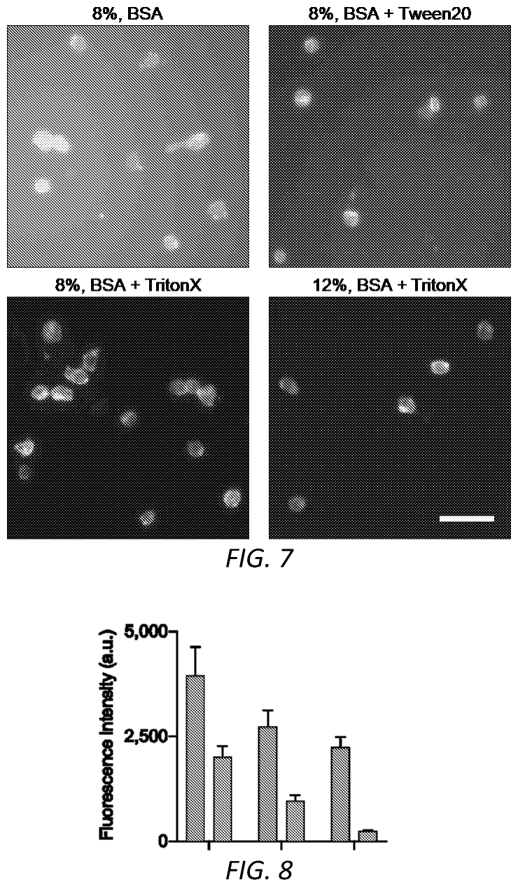

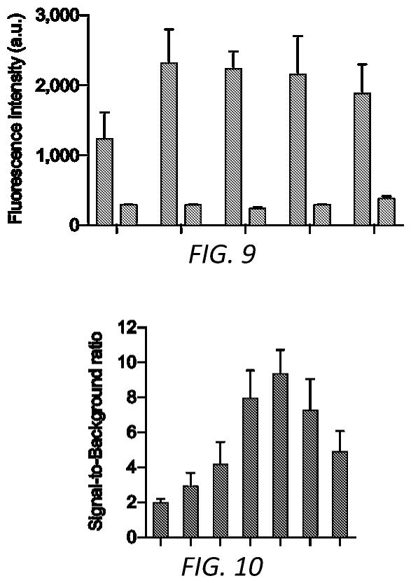

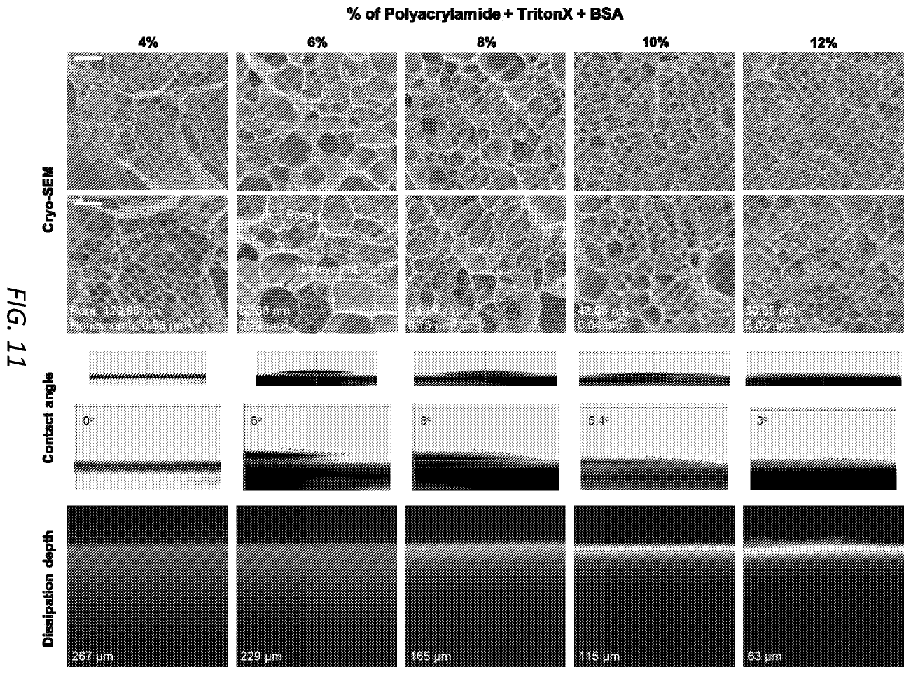

Hydrogel stamps were made in different hydrogel concentrations and surfactant additives to optimize a wash-free immunostaining condition. Bovine serum albumin (BSA) was added to reduce the nonspecific adsorption of antibodies to the hydrogel. Adding Tween 20 or Triton X-100 significantly increased the hydrophilicity of the hydrogel. The hydrogels' contact angles were reduced from 64° when only BSA was added, to 9° for BSA with Tween 20, and 8° for BSA with Triton X-100 (FIG. 6). The difference in the hydrophilicity affected the background signals after immunostaining. FIG. 7 shows representative images of BT474 breast cancer cells stained for HER2 using different hydrogel stamps. Significant background fluorescence signals outside of cells were observed when the hydrogel with only BSA was used, which would require a washing step to wash out unbound antibodies (FIG. 8). In contrast, the background signal was significantly lower when a hydrogel with BSA and Triton X-100 was used. Without intending to be bound by any particular theory, this may be due to the highest hydrophilicity of hydrogel with Triton X-100, leaving a negligible amount of unbound antibodies on the substrate. For different concentrations of hydrogels, 8% hydrogel with BSA and Triton X-100 showed the highest signal-to-background ratio (FIGS. 9-10). In the cryo-SEM analysis, hydrogel with a lower concentration of polyacrylamide was observed to have a larger pore size (FIG. 11). When a liquid solution was dropped on the surface of the hydrogel, the liquid was completely (4%) or partially (6%) absorbed into the hydrogel within 5 sec with longer dissipation depths. This might lead to inefficient immunolabeling whereby most of the antibodies will be absorbed into the hydrogel before staining. On the contrary, for 10% and 12% hydrogels with smaller pore sizes, no absorption was observed over 10 sec with short dissipation depths. In such a case, unbound antibodies after staining might not be efficiently removed by the hydrogel, leading to higher background signals. Therefore, 8% polyacrylamide was an optimal concentration for efficient immunostaining and removal of unbound antibodies.

### Optimization of Immunostaining of Cells

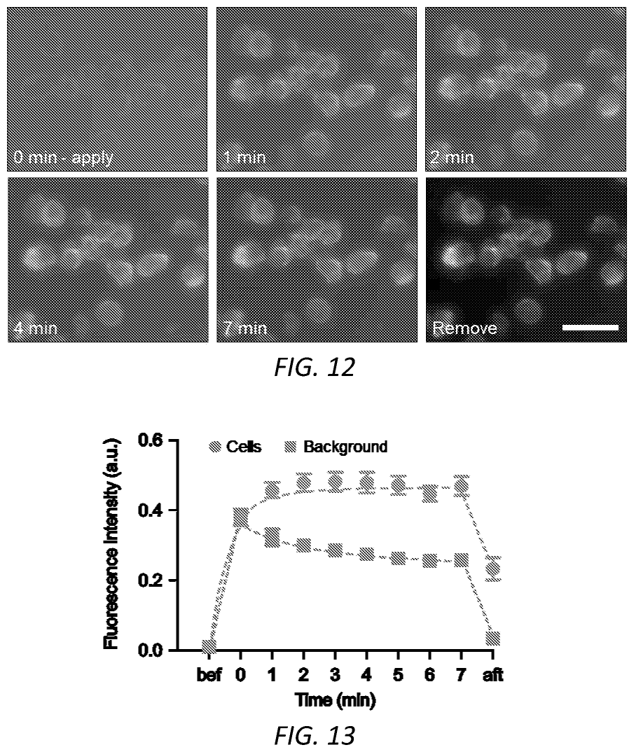

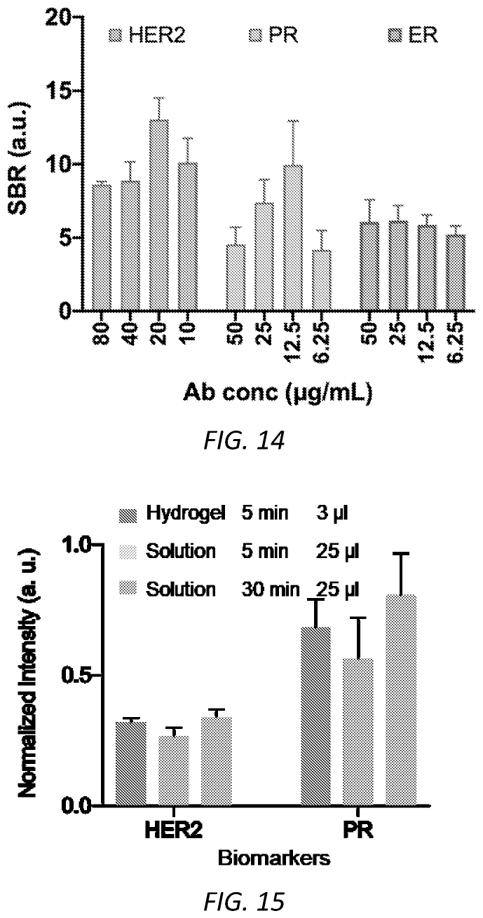

Next, hydrogel immunostaining was optimized and evaluated. The optimal staining time was investigated by monitoring the staining kinetics. FIGS. 12-13 show fluorescence images and mean fluorescence intensities of BT474 cells and surrounding backgrounds during hydrogel stamping with fluorescent HER2 antibodies. The results show that immunostaining reached saturation around 5 min. after the hydrogel was applied. When the hydrogel was detached from the surface, unbound antibodies were removed, and the background signals went down <0.03 while positive HER2 signals on cells remained above 0.2. Optimal antibody concentrations were also tested, and found to be in the range of 10-20 μg/mL (FIG. 14).

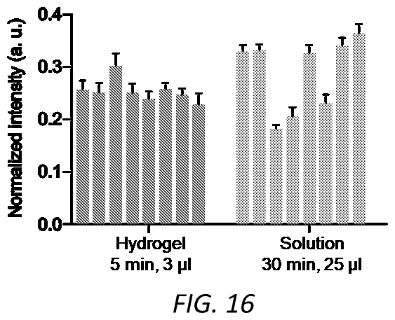

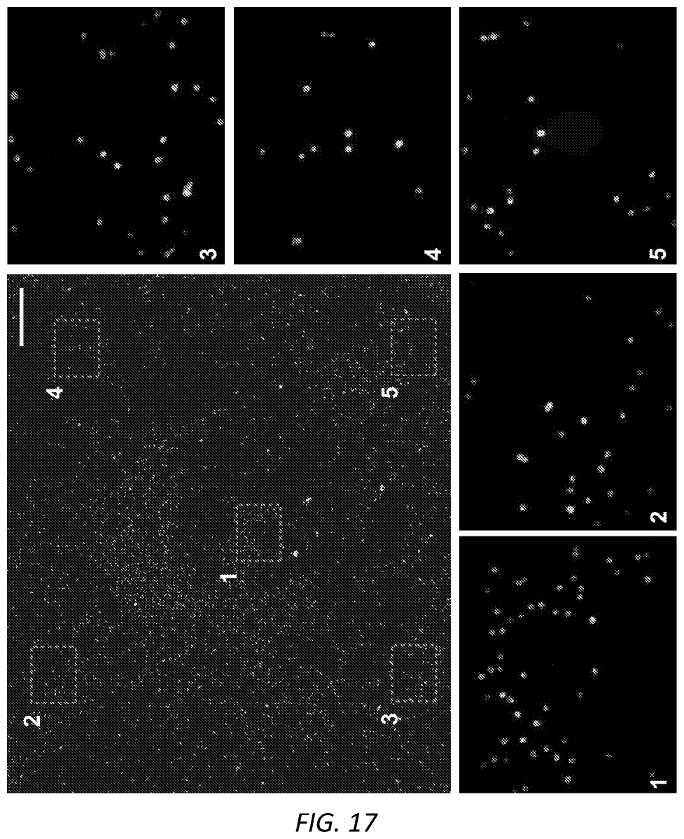

The staining efficiency between hydrogen stamping and conventional solution-based incubation was compared. BT474 cells were labeled for HER2 as a surface marker and PR as an intracellular marker through i) 5 min. hydrogel stamping with 3 μL antibody; ii) 5 min. incubation with 25 μL antibody solution; and iii) 30 min. incubation with 25 μL antibody solution. The signal intensities from the hydrogel method were higher than those from the 5 min. solution-based method and comparable to those from the 30 min. solution-based method (FIG. 15). These highlight the hydrogel method's advantages in terms of shorter labeling time (5 min) and lower reagent volume (3 μL). Moreover, the hydrogel approach shows excellent staining reproducibility and uniformity. Eight repeated labeling experiments were conducted for both 30 min. solution-based method and the 5 min. hydrogel method (FIG. 16). In the conventional solution-based approach, the coefficient of variation (CV) was 24.5%, whereas the CV value was 8.6% in the hydrogel method. FIG. 17 shows the uniform labeling of fixed cells in a 1 cm×1 cm region using hydrogel stamping.

### Example 2. Immunocycling

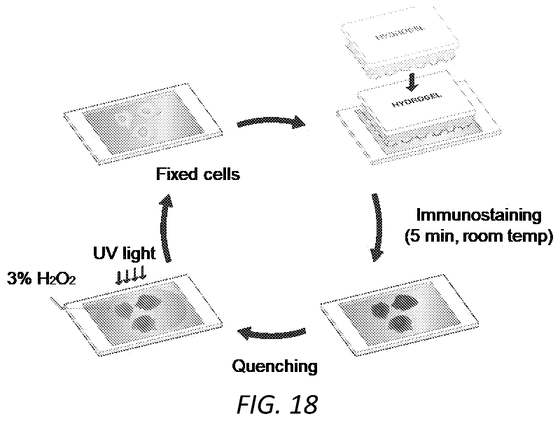

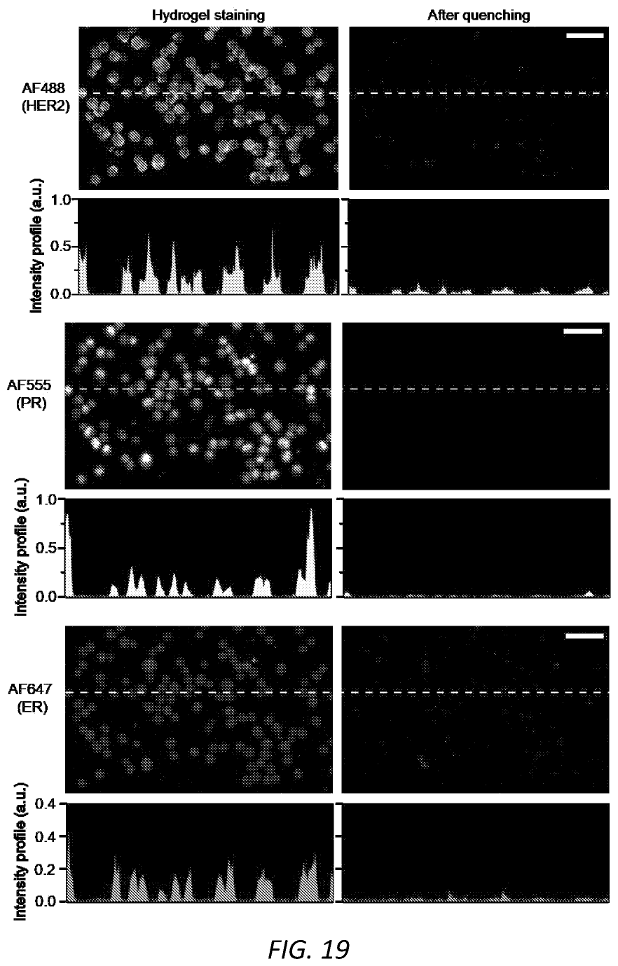

Immunocycling was implemented using hydrogel staining to profile 9 different cancer biomarkers on the same population of cells. As a first step, existing fluorophores were quenched after immunolabeling and cells were relabeled with another set of antibodies. Hydrogen peroxide (H₂O₂) was used as a quenching solution, which was applied onto the cells directly and catalyzed by illuminating with an ordinary incandescent lamp for 5 min. Combined with 5 min. hydrogel immunostaining and fluorescence imaging, each cycle took less than 15 min. for cellular samples. BT474 breast cancer cells were labeled against HER2, PR, and ER primary antibodies conjugated with AF488, AF555, and AF647 fluorophores, respectively. Subsequently, the labeled cells were quenched using 5% hydrogen peroxide and 7.5% sodium bicarbonate solution. FIG. 18 shows a workflow of the immunocycling of fixed cells. FIG. 19 shows fluorescence signals after immunostaining and quenching, illustrating effective quenching of fluorescence signals.

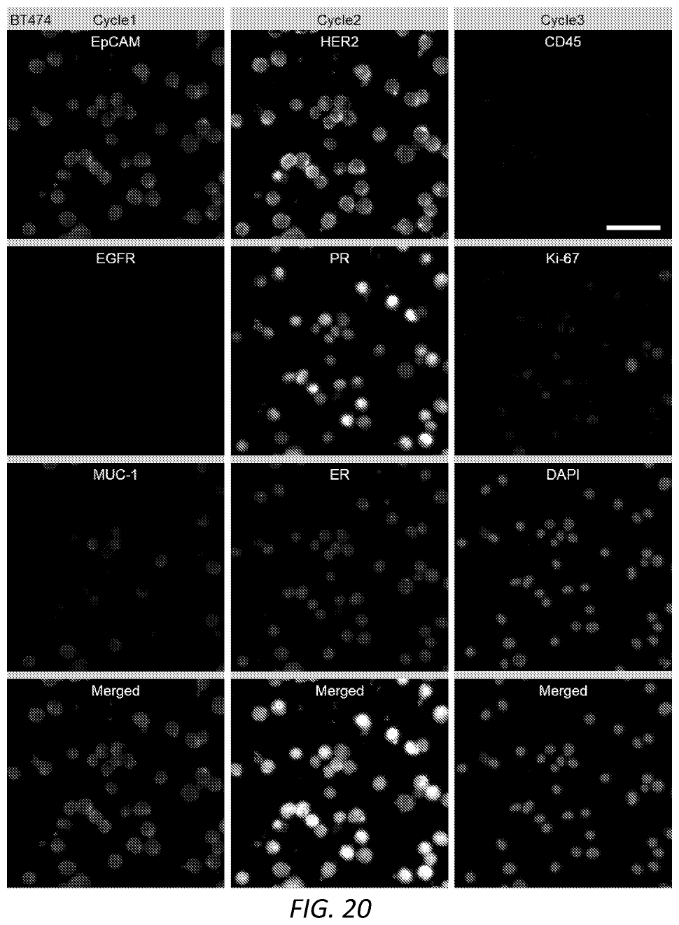

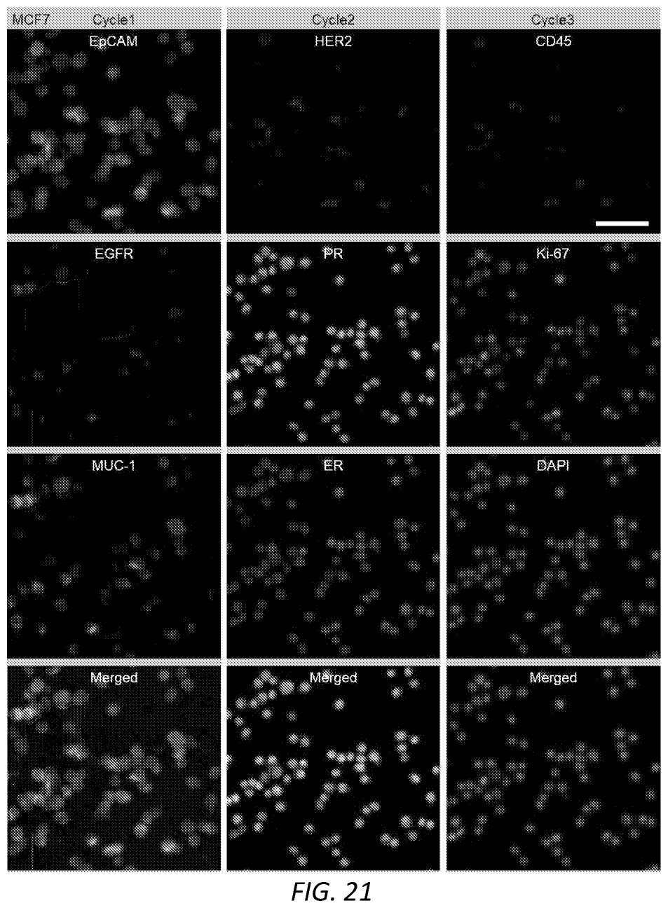

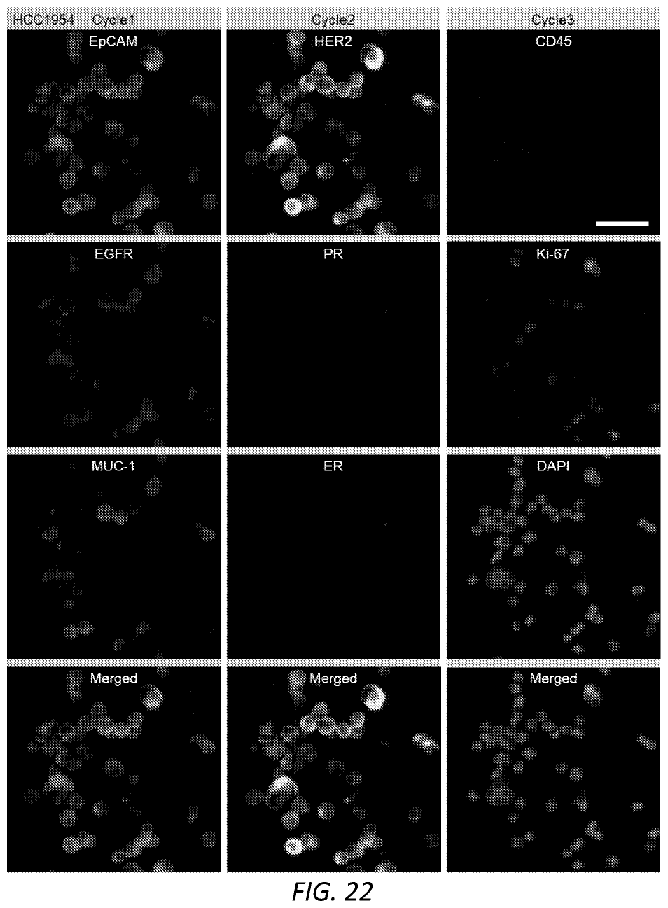

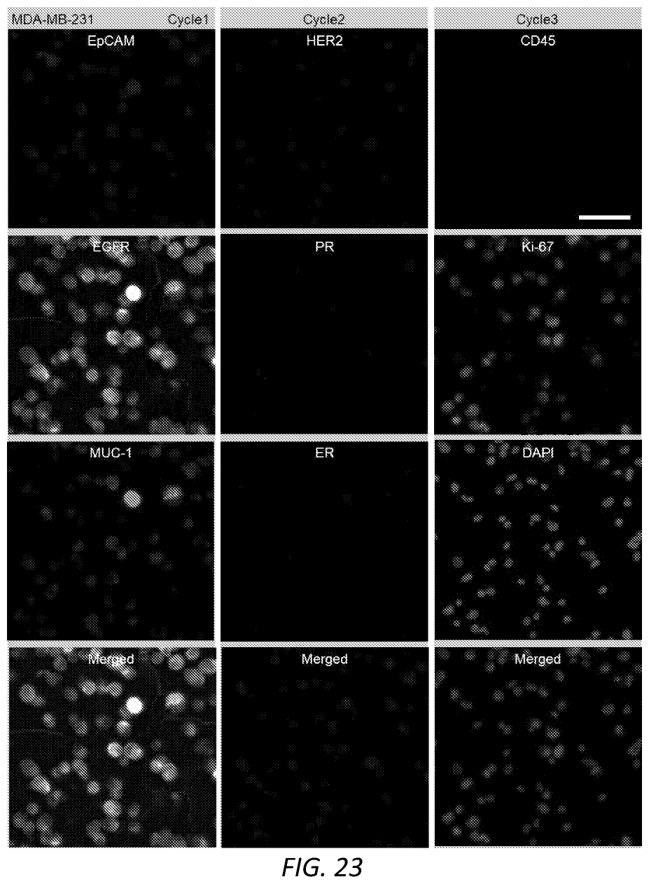

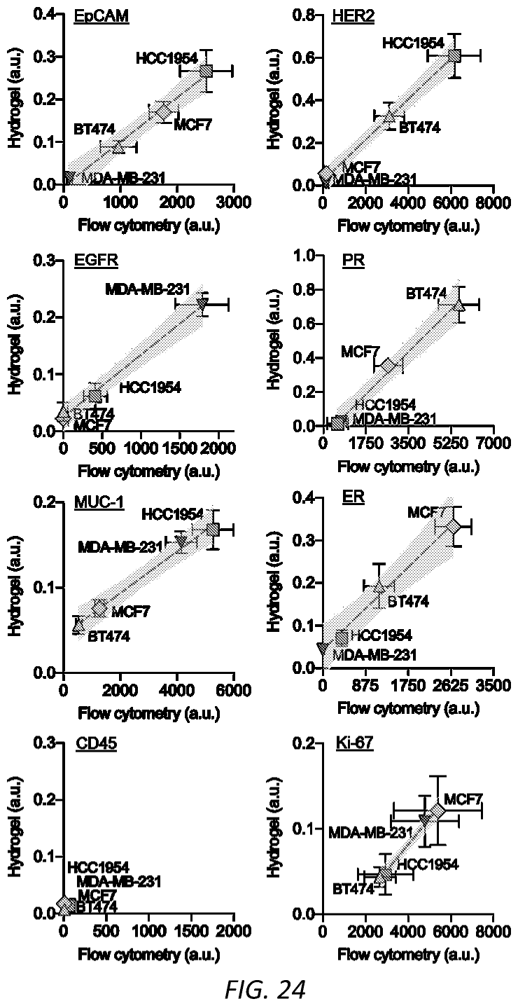

Next, the hydrogel-based immunocycling was applied for molecular profiling of cancer cell lines against 9 biomarkers, including EpCAM, EGFR, and MUC-1 for cancer cell detection, HER2, PR, and ER for subtyping, Ki-67 as a proliferative marker, CD45 for immune cells, and DAPI for nuclei staining. Four breast cancer cell lines that represented different breast subtypes-BT474 (HER2, PR, and ER positive), HCC1954 (HER2 positive, ER, and PR negative), MCF7 (HER2 negative, PR, and ER positive), and MDA-MB-231 (triple negative)-were tested. FIG. 20 shows fluorescence images of labeled BT474 cells, showing positive signals for EpCAM, HER, PR, and ER as a triple-positive breast cancer cell line. The fluorescence images of immunocycling of MCF7, HCC1954, and MDA-MB-231 are shown in FIGS. 21-23. The cellular fluorescence intensities obtained from hydrogel immunostaining were compared with those from the gold standard flow cytometry (FIG. 24). The comparison shows excellent correlations (R²>0.99) of the mean fluorescence intensities between hydrogel immunostaining and flow cytometry methods for target markers, showing the quality staining and cycling using hydrogels. However, immunostaining by hydrogel excelled in operation; it required much smaller sample volumes and led to faster staining time without extra washing steps than conventional solution-based immunolabelling.

### Example 3. Immunocycling of Tissue Samples

Next, hydrogel immunocycling was applied for multiplexed molecular analysis of breast cancer tissue samples. Analysis of formalin-fixed paraffin-embedded (FFPE) tissue specimens has been the gold standard procedure in clinical pathology laboratories for research, examination, diagnosis, and drug development. IHC is the most common procedure for FFPE specimens to measure target protein expression levels in tumors and other adjacent cells. Conventional IHC on tissues normally requires overnight incubation of primary antibodies in 4° C. and is often limited to a single marker per tissue slide.

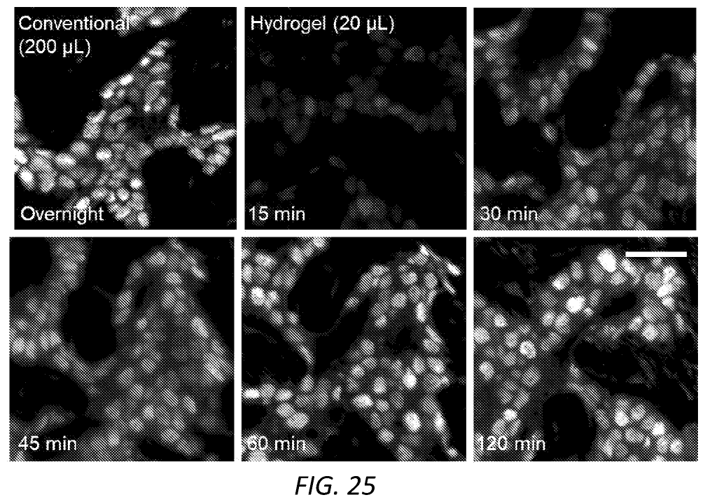

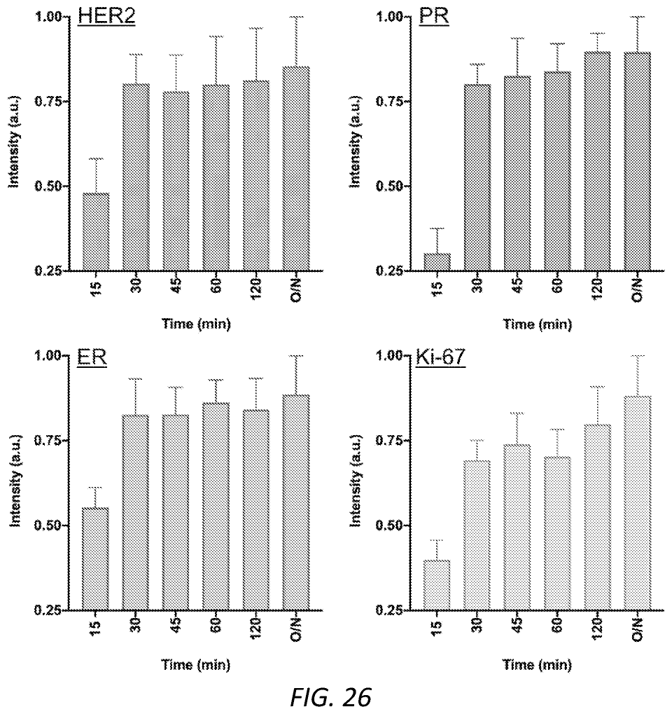

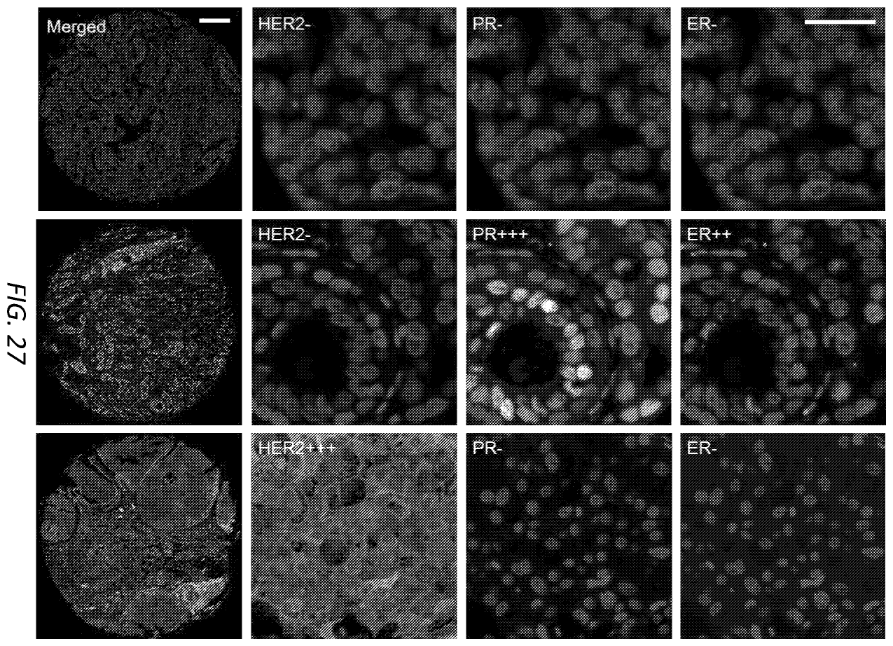

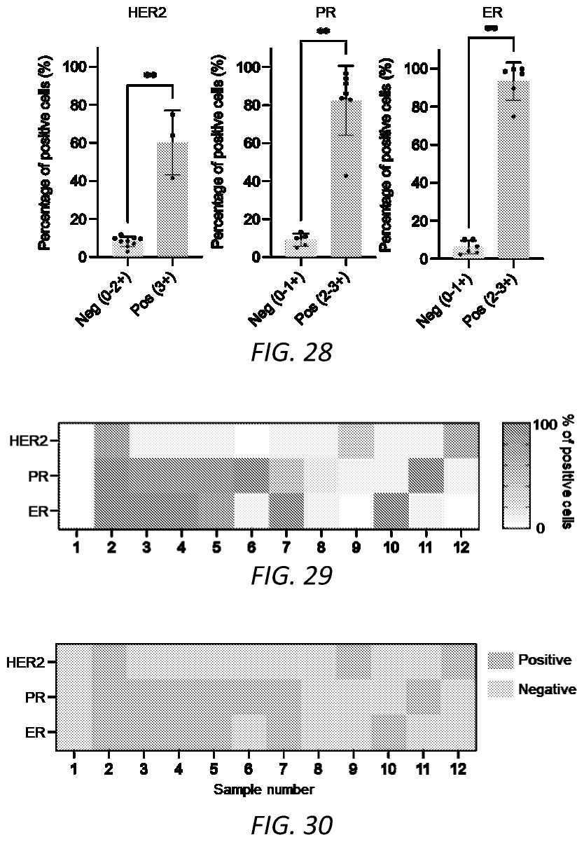

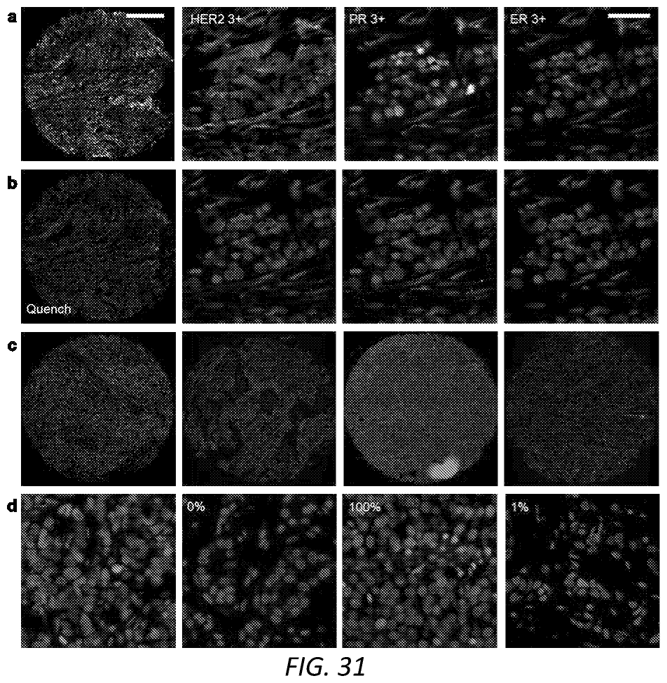

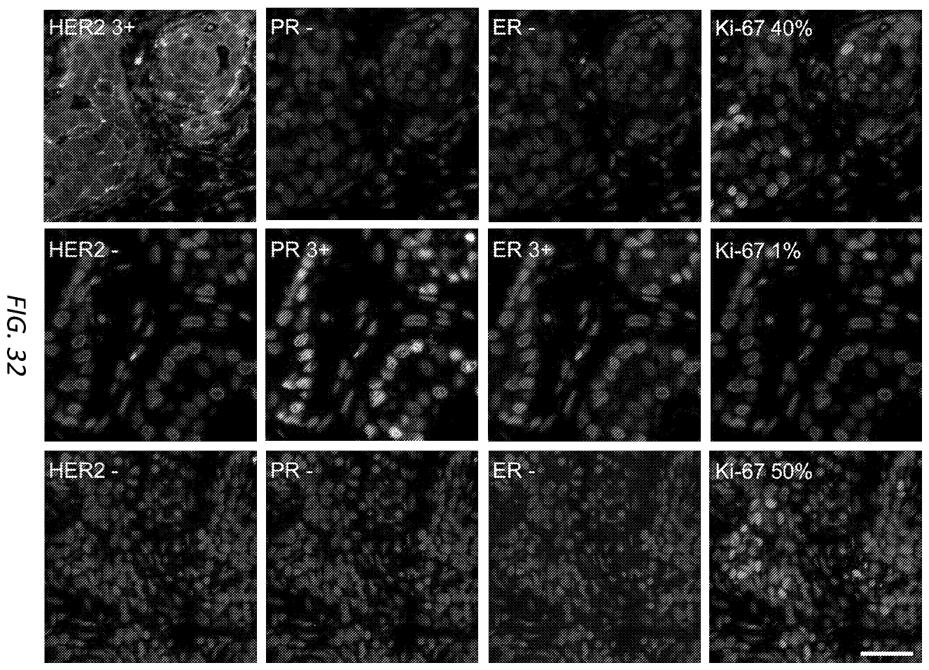

First, tissue sections were stained with positive expression of target markers (HER2, PR, ER, and Ki-67) overnight based on conventional protocol with 200 μl antibody solutions and compared fluorescence intensities with tissue sections stained by hydrogels for 15, 30, 45, 60, and 120 min. using 20 μl antibody solutions (FIG. 25). In general, the incubation time of 30 min. using hydrogels was sufficient to stain the respective markers with a mean intensity of more than 80% compared to overnight incubation in the conventional method (FIG. 26). Therefore, the 30 min. incubation time was used for subsequent immunostaining of breast cancer tissues. Hydrogel immunostaining was tested on 12 tissue samples with different molecular subtypes of HER2, PR, and ER. Results are shown in FIGS. 27-32.

The results show that immunostaining using a contact-type patch can be highly efficient and simple for cells and tissue samples. Immunocycling using a contact-type patch can provide rapid, multiplexed molecular profiling of cellular and tissue specimens, potentially applicable for cancer diagnostics, immune cell profiling, therapeutic efficacy, and drug screening.

### Example 4. Fluorescence Quenching

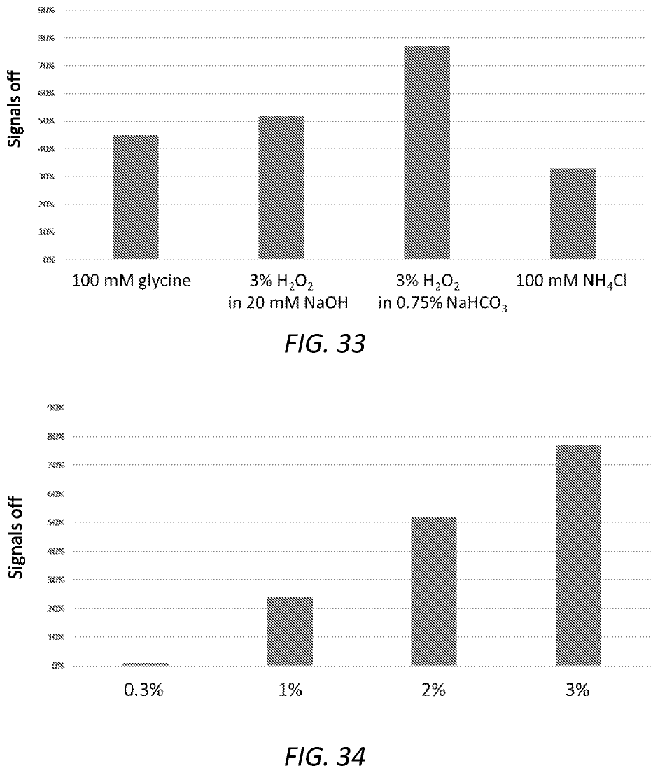

Quenching agents were evaluated for a multiplexed assay. As shown in FIG. 33, 3% H₂O₂ in 0.75% NaHCO₃ showed improved quenching as compared to 100 mM glycine, 3% H₂O₂ in 20 mM NaOH, and 100 mM NH₄Cl. As shown in FIG. 34, quenching by H₂O₂ improved with increasing concentration.

### Example 5. Hydrogel Quenching

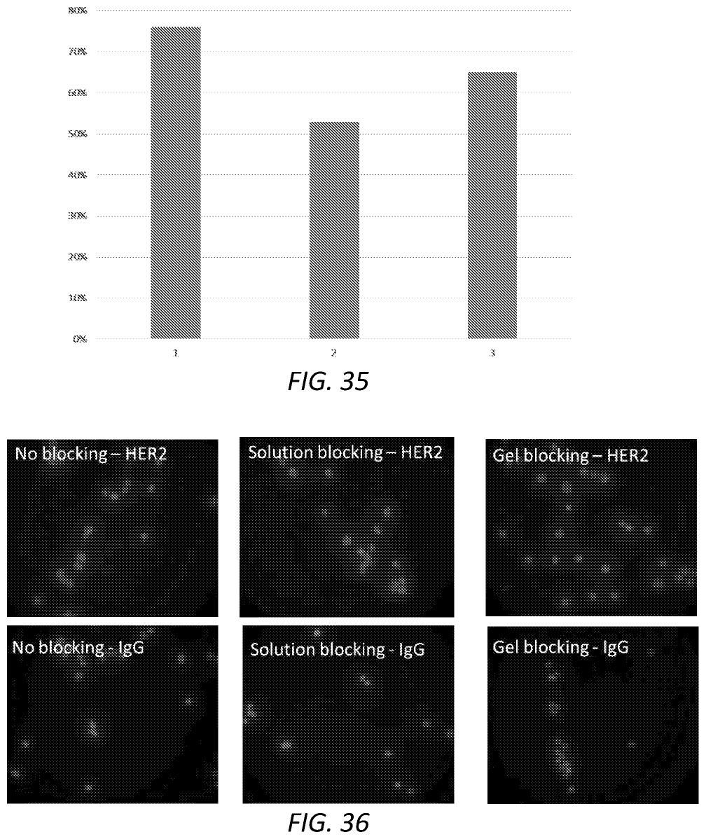

10 μL of 3% H₂O₂ in 0.75% NaHCO₃ was added to an acrylamide hydrogel prepared as discussed above, which was then applied to an immunostained sample for a period of 10 min., 20 min., or 30 min. As shown in FIG. 35, quenching efficiency was best after a 10-minute contact period. The signal-off level resulting from a 10-minute contact period with the H₂O₂-containing hydrogel was comparable to that achievable with a solution-based approach.

### Example 6. Hydrogel Blocking and Permeabilization

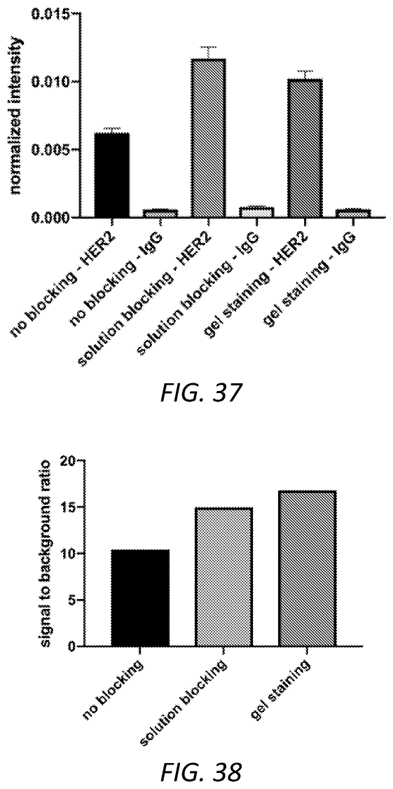

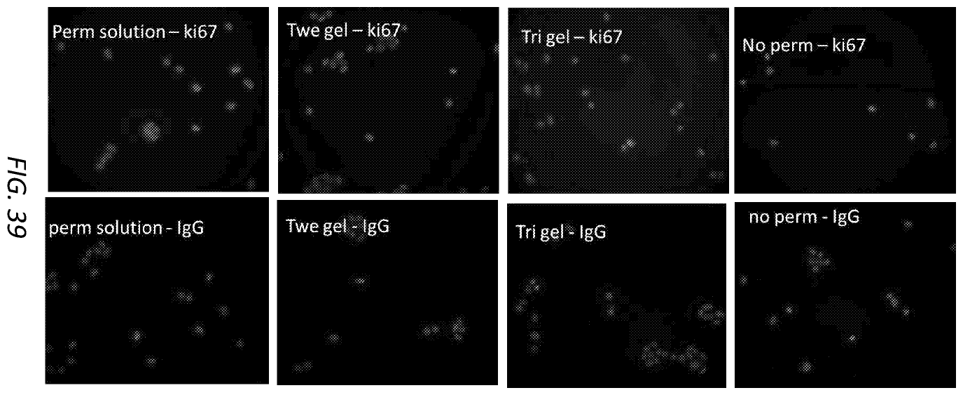

Immunostaining for HER2 and IgG according to Example 2 was conducted (i) using a hydrogel stamp free from blocking agents, for a sample of un-blocked cells, (ii) using a hydrogel stamp free from blocking agents, for a sample of cells previously blocked using 2.5% BSA (37525, ThermoFisher Scientific) and 2.5% goat serum (5425S, Cell Signaling Technology) in BD Perm/Wash buffer for 10 min, and (iii) using a hydrogel stamp including 1 wt % BSA (37525, ThermoFisher Scientific), for a sample of un-blocked cells. As shown in FIGS. 36-38, the intensity and normalized intensity of cells blocked using a BSA-containing hydrogel were comparable to that achievable with a solution-based approach.

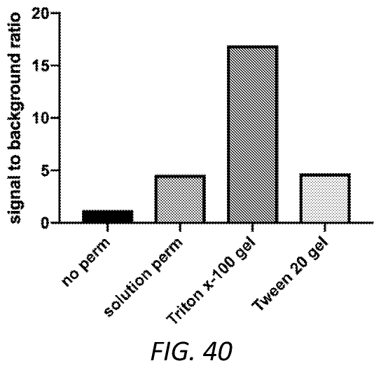

Immunostaining for ki67 and IgG according to Example 2 was conducted (i) using a hydrogel stamp free from surfactant, for a sample of cells previously permeabilized using BD Perm/Wash buffer (554723, BD Biosciences) for 10 min, and (ii) using a hydrogel stamp including 0.1 wt % Tween 20, for a sample of un-permeabilized cells, and (iii) using a hydrogel stamp including 0.1 wt % Triton X-100, for a sample of un-permeabilized cells. As shown in FIGS. 39-40, the signal-to-background ratio of cells permeabilized using a Tween 20-containing gel was comparable to that achievable with a solution-based approach, and the signal-to-background ratio of cells permeabilized using a Triton X-100-containing gel was better than that achievable with a solution-based approach.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of quenching a fluorophore present in a biological sample, the method comprising contacting a surface of a quenching patch with the sample, wherein the quenching patch comprises:
a substrate comprising a polymer; and
a quenching agent disposed within the substrate.

2. The method of claim 1, wherein the polymer comprises a covalently cross-linked polymer.

3. The method of claim 2, wherein the polymer comprises acrylamide and bis-acrylamide monomer units.

4. The method of claim 1, wherein the polymer comprises a polysaccharide.

5. The method of claim 4, wherein the polymer comprises agarose.

6. The method of claim 1, wherein the quenching patch comprises:
about 4 wt % to about 12 wt % of the polymer; and
about 88 wt % to about 96 wt % of water.

7. The method of claim 1, wherein the quenching patch comprises:
about 6 wt % to about 10 wt % of the polymer; and
about 90 wt % to about 94 wt % of water.

8. The method of claim 1, wherein the polymer comprises a polymer network comprising a plurality of pores.

9. The method of claim 8, wherein an average pore size of the plurality of pores is about 20 nm to about 75 nm.

10. The method of claim 8, wherein an average pore size of the plurality of pores is about 30 nm to about 65 nm.

11. The method of claim 1, wherein the quenching agent is capable of oxidizing the fluorophore.

12. The method of claim 11, wherein the quenching agent comprises a peroxide group, a periodate group, or a mixture thereof.

13. The method of claim 11, wherein the quenching agent comprises hydrogen peroxide.

14. The method of claim 11, wherein the polymer comprises the peroxide group or periodate group.

15. The method of claim 1, wherein the quenching agent comprises an iodide, an acrylamide, a black hole quencher, a QSY quencher, a DABCYL quencher, malachite green, gold nanoparticles, graphene, or any mixture thereof.

16. The method of claim 1, wherein the quenching patch further comprises a surfactant disposed within the substrate.

17. The method of claim 16, wherein the surfactant is selected from a polysorbate, an octylphenol ethoxylate, a saponin, formaldehyde, methanol, and mixtures thereof.

18. The method of claim 16, wherein the surfactant is selected from Tween 20, Triton X-100, and mixtures thereof.

19. The method of claim 16, wherein the quenching patch comprises about 0.01 wt % to about 2 wt % of the surfactant.

20. The method of claim 1, wherein the quenching patch further comprises a blocking agent disposed within the substrate.

21. The method of claim 20, wherein the blocking agent is selected from bovine serum albumin, fetal bovine serum, steelhead salmon serum, non-fat milk, SuperBlock™, AdvanBlock™, and mixtures thereof.

22. The method of claim 20, wherein the blocking agent is bovine serum albumin.

23. The method of claim 20, wherein the quenching patch comprises about 0.1 wt % to about 5 wt % of the blocking agent.

24. The method of claim 1, wherein a water contact angle of the surface of the quenching patch is less than about 60°.

25. The method of claim 1, wherein a water contact angle of the surface of the quenching patch is about 5° to about 20°.

26. The method of claim 1, wherein a water contact angle of the surface of the quenching patch is less than a water contact angle of a surface of the biological sample.

27. The method of claim 1, further comprising:
providing a precursor patch comprising the susbtrate; and then
applying an aqueous solution comprising the quenching agent to a surface of the substrate to form the quenching patch.

28. The method of claim 27, wherein the aqueous solution comprises about 0.5 wt % to about 10 wt % of the quenching agent.

29. The method of claim 27, wherein the aqueous solution comprises about 1 wt % to about 5 wt % of the quenching agent.

30. The method of claim 27, wherein a pH of the aqueous solution is less than 6.

31. The method of claim 27, wherein a pH of the aqueous solution is about 7 to about 10.

32. The method of claim 27, comprising applying about 1 μL to about 20 μL of the aqueous solution per square centimeter of the surface of the precursor patch.

33. The method of claim 27 comprising applying about 2 μL to about 10 μL of the aqueous solution per square centimeter of the surface of the precursor patch.

34. The method of claim 1, wherein the fluorophore comprises a fluorescence-generating protein or small molecule.

35. The method of claim 1, wherein the fluorophore comprises a coumarin dye, a rhodamine dye, a cyanine dye, or a xanthene dye.

36. The method of claim 1, wherein the sample comprises cells selected from tumor cells, non-tumor cells, immune cells, host cells, blood cells, and mixtures thereof.

37. The method of claim 36, wherein the cells comprise tissue.

38. The method of claim 1, wherein the sample comprises tissue comprising tumor cells.

39. A method of staining a biological sample, the method comprising:
contacting a surface of a first staining patch with the sample, wherein the first staining patch comprises:
a substrate comprising a polymer; and
a first fluorophore-conjugated probe disposed within the substrate;
removing the first staining patch to provide a first stained sample;
measuring a fluorescence intensity of the first stained sample;
contacting a surface of a quenching patch with the first stained sample, wherein the quenching patch comprises:
a substrate comprising a polymer; and
a quenching agent disposed within the substrate;
removing the quenching patch to provide a quenched sample;
contacting a surface of a second staining patch with the quenched sample, wherein the second staining patch comprises:
a substrate comprising a polymer; and
a second fluorophore-conjugated probe disposed within the substrate;
removing the second staining patch to provide a second stained sample; and
measuring a fluorescence intensity of the second stained sample.

40. The method of claim 39, wherein at least 70 wt % of an amount of the first fluorophore-conjugated probe present in the first stained sample is bound to a first biomarker.

41. The method of claim 39, wherein a contribution of the first fluorophore-conjugated probe to the fluorescence intensity of the second stained sample is no more than 20%.

42. The method of claim 39, wherein at least 70 wt % of an amount of the second fluorophore-conjugated probe present in the second stained sample is bound to a second biomarker.

43. The method of claim 39, wherein an absorbance spectrum of the first fluorophore-conjugated probe and an absorbance spectrum of the second fluorophore-conjugated probe overlap.

44. The method of claim 39, wherein one or more of the first staining patch, the quenching patch, and the second staining patch independently comprise:
about 4 wt % to about 12 wt % of the polymer; and
about 88 wt % to about 96 wt % of water.

45. The method of claim 39, wherein one or more of the first staining patch, the quenching patch, and the second staining patch independently comprise:
about 4 wt % to about 12 wt % of the polymer;
about 88 wt % to about 96 wt % of water;
about 0.01 wt % to about 1 wt % of a surfactant; and
about 0.1 wt % to about 3 wt % of the blocking agent.

46. The method of claim 44, wherein the polymer comprises a covalently cross-linked polymer comprising acrylamide and bis-acrylamide monomer units.

47. The method of claim 44, wherein the polymer network comprises agarose.

48. The method of claim 39, wherein one or both of the first fluorophore-conjugated probe and the second fluorophore-conjugated probe comprise a coumarin dye, a rhodamine dye, a cyanine dye, or a xanthene dye.

49. The method of claim 39, wherein the sample comprises cells selected from tumor cells, non-tumor cells, immune cells, host cells, blood cells, and mixtures thereof.

50. The method of claim 49, wherein the cells comprise tissue.

51. The method of claim 39, wherein the sample comprises tissue comprising tumor cells.

52. The method of claim 39, wherein one or both of the first fluorophore-conjugated probe and the second fluorophore-conjugated probe comprise an antibody.

53. The method of claim 39, wherein one or both of the first fluorophore-conjugated probe and the second fluorophore-conjugated probe comprise an antibody independently capable of binding to a biomarker selected from cancer biomarkers, neurodegenerative disease biomarkers, blood/vascular disease biomarkers, infection biomarkers, inflammation biomarkers, and wound-healing biomarkers.

54. The method of claim 39, wherein one or both of the first fluorophore-conjugated probe and the second fluorophore-conjugated probe comprise an antibody independently capable of binding to a biomarker selected from cancer biomarkers.

55. The method of claim 39, wherein one or both of the first fluorophore-conjugated probe and the second fluorophore-conjugated probe comprise an antibody independently capable of binding to a biomarker selected from EpCAM, EGFR, MUC-1, HER2, PR, ER, Ki-67, CD45, and DAPI.

56. The method of claim 39, wherein one or both of the first fluorophore-conjugated probe and the second fluorophore-conjugated probe comprise a nucleic acid.

57. A kit, comprising
a precursor patch comprising a substrate comprising a polymer;
a first aqueous solution comprising a first fluorophore-conjugated probe;
a second aqueous solution comprising a second fluorophore-conjugated probe; and
a third aqueous solution comprising a quenching agent.

58. The kit of claim 56, wherein the precursor patch comprises:
about 4 wt % to about 12 wt % of the polymer; and
about 88 wt % to about 96 wt % of water.

59. The kit of claim 56, wherein the precursor patch comprises:
about 4 wt % to about 12 wt % of the polymer;
about 88 wt % to about 96 wt % of water;
about 0.01 wt % to about 1 wt % of a surfactant; and
about 0.1 wt % to about 3 wt % of the blocking agent.

60. The kit of claim 56, wherein the polymer comprises a polymer network comprising a plurality of pores.

61. The kit of claim 56, wherein the polymer comprises a covalently cross-linked polymer comprising acrylamide and bis-acrylamide monomer units.

62. The kit of claim 56, wherein the polymer comprises agarose.

63. The kit of claim 56, wherein the first fluorophore-conjugated probe and the second fluorophore-conjugated probe each independently comprise a fluorophore-conjugated antibody.
